(19) United States
(12) Patent Application Publication
(10) Pub. No.: US 2023/0330269 A1
(43) Pub. Date: Oct. 19, 2023

(54) TREATMENT OF RPE65-ASSOCIATED EYE DISEASES AND DISORDERS

(71) Applicant: HuidaGene Therapeutics Co., Ltd., Shanghai (CN)

(72) Inventor: Shaoran Wang, Shanghai (CN)

(21) Appl. No.: 18/047,255

(22) Filed: Oct. 17, 2022

Related U.S. Application Data
(63) Continuation of application No. PCT/CN2022/117383, filed on Sep. 6, 2022.

(30) Foreign Application Priority Data
Sep. 6, 2021 (WO) PCT/CN2021/116781

(51) Int. Cl. A61K 48/00; C12N 15/86; A61P 27/02

(52) U.S. Cl. CPC A61K 48/0066; C12N 15/86; A61P 27/02; C12N 2750/14143; C12N 2750/14145; C12N 2750/14171; C12N 2750/14122; C12N 2830/50

(57) ABSTRACT

The disclosure described herein provides recombinant adeno-associated virus (rAAV) viral particle comprising an AAV9 serotype capsid and a vector genome encoding an RPE65 (e.g., hRPE65) polypeptide, as well as related compositions and uses thereof (e.g., use to treat Leber's congenital amaurosis 2 (LCA2)).

### REFERENCE TO RELATED APPLICATION

This application is a continuation application, filed under 35 U.S.C. 111(a), of International Patent Application No. PCT/CN2022/117383, filed Sep. 6, 2022, which claims priority to International Patent Application No. PCT/CN2021/116781, filed on Sep. 6, 2021, the entire content of each of which, including all drawings and sequence listings, are incorporated herein by reference.

### REFERENCE TO AN ELECTRONIC SEQUENCE LISTING

The instant application contains a Sequence Listing XML file which has been submitted electronically in ASCII format, and is hereby incorporated by reference in its entirety. Said Sequence Listing XML file, created on Oct. 11, 2022, is named 132045-00901.xml, and is 23,231 bytes in size.

### BACKGROUND

About 1 in 2,300 people in the world suffers from inherited retinal diseases (IRDs) caused by various genes but rarely have effective treatment. Mutations in more than 300 single genes have been found to cause the loss of a functional protein that affects visual function. Most of these functional genes were expressed in the retinal pigment epithelium (RPE) and photo receptor cells. Their dysfunction or lack may cause some IRDs, such as: Retinitis pigmentosa, Stargardt's disease, choroiditis, and Leber's congenital amaurosis (LCA).

LCA disease, first reported by German ophthalmologist Theodor Leber in 1869, is one of the earliest and most severe hereditary retinopathy and people suffered from LCA have severe visual function impairment beginning in infancy. LCA accounts for more than 5% of inherited retinopathy and is the leading cause of congenital blindness in children (10% to 20%). LCA is clinically characterized by nystagmus, sluggish or near-absent pupillary responses, severely decreased visual acuity, photophobia, and high hyperopia.

LCA diseases are mostly inherited autosomal recessive and there are at least 22 known pathogenic genes at present. LCA disease caused by the mutation of RPE65 gene is called LCA2 (Leber's congenital amaurosis 2), accounting for about 6%-15% of the total LCA. RPE65, with a size of 65 kD, is mainly expressed in retinal pigment epithelial cells, also known as retinoid pigment isomerase. RPE65 protein catalyzes the isomerization of all-trans retinol into 11-cis-retinol in the visual cycle, which plays an important role in maintaining visual function. Mutation of RPE65 leads to the accumulation of all-trans retinol and the inability to regenerate rhodopsin, resulting in visual dysfunction.

At present, the treatment for LCA2 is mainly gene therapy. AAV was used as a vector to deliver the correct RPE65 gene to target cells to restore visual function. In 2017, the FDA approved LUXTURNA® (voretigene neparvovec-rzyl), the first gene therapy drug, for the treatment of LCA2. The clinical results of LUXTURNA® (voretigene neparvovec-rzyl), a gene therapy drug with AAV2 serotype (i.e., AAV2 capsid) carrying the human RPE65 gene, showed improvement in vision. However, as reported, the visual function restored by LUXTURNA® (voretigene neparvovec-rzyl) still have room for further improvement.

Thus, there is a need to provide additional gene therapy to treat RPE65-associated eye disease and disorders.

### SUMMARY

One aspect of the disclosure provides a recombinant adeno-associated virus (rAAV) viral particle comprising an AAV9 serotype capsid and a vector genome encoding an RPE65 (e.g., hRPE65) polypeptide, such as a polynucleotide at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO: 1.

A related aspect of the disclosure provides a recombinant adeno-associated virus (rAAV) vector genome encoding an RPE65 (e.g., hRPE65) polypeptide, such as a polynucleotide having a sequence identity of at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% to SEQ ID NO: 1.

In certain embodiments, the rAAV vector genome comprises: a) a 5′ inverted terminal repeat (ITR); b) an RPE65 polynucleotide encoding an RPE65 polypeptide, wherein the RPE65 polynucleotide comprises a polynucleotide sequence of SEQ ID NO: 2 or having a sequence identity of at least 90%, 95%, 96%, 97%, 98%, 99%, 99.2%, 99.4%, 99.6%, or 99.8% to the polynucleotide sequence of SEQ ID NO: 2; c) a promoter operably linked to and drives the transcription of the RPE65 polynucleotide; d) an optional Kozak sequence upstream of the RPE65 polynucleotide and downstream of the promoter; e) a polyA signal sequence; and f) a 3′ ITR. In certain embodiments, the RPE65 polypeptide has the amino acid sequence of SEQ ID NO: 3.

In certain embodiments, the 5′ ITR and 3′ ITR are derived from AAV2 or AAV9. In certain embodiments, the 5′ ITR comprises the nucleotide sequence of SEQ ID NO: 7. In certain embodiments, the 3′ ITR comprises the nucleotide sequence of SEQ ID NO: 8.

In certain embodiments, the promoter is a ubiquitous promoter. In certain embodiments, the promoter is a tissue-specific promoter. In certain embodiments, the promoter is a constitutive promoter. In certain embodiments, the promoter is an inducible promoter.

In certain embodiments, the promoter is selected from the group consisting of a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (IE) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nP2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, or a functional fragment thereof.

In certain embodiments, the promoter is the CAG promoter.

In certain embodiments, the CAG promoter comprises a sequence having a sequence identity of at least 95%, 96%, 97%, 98%, or 99% to SEQ ID NO: 4.

In certain embodiments, the CAG promoter comprises, consists essentially of, or consists of, SEQ ID NO: 4.

In certain embodiments, the polyA signal sequence is selected from the group consisting of bovine growth hormone polyadenylation signal sequence (bGH polyA), a small polyA signal sequence (SPA), a human growth hormone polyadenylation signal sequence (hGH polyA), a rabbit beta globin polyA signal sequence (rBG polyA), a SV40 polyA signal sequence (SV40 polyA), or a variant thereof. In certain embodiments, the polyA signal sequence is the bGH polyA.

In certain embodiments, the bGH polyA comprises, consists essentially of, or consists of, SEQ ID NO: 5.

In certain embodiments, the Kozak sequence is GCCACC (SEQ ID NO: 6); or a sequence comprising at most 1, 2, 3, or 4 nucleotide differences from GCCACC (SEQ ID NO: 6), and optionally wherein the last three nucleotide is ACC or GCC.

In certain embodiments, the vector genome comprises, in 5′ to 3′ direction,

(1) a 5′ ITR of SEQ ID NO: 7,

(2) a CAG promoter of SEQ ID NO: 4,

(3) a Kozak sequence of GCCACC (SEQ ID NO: 6),

(4) a hRPE65 polynucleotide sequence of SEQ ID NO: 2,

(5) a bGH polyA signal sequence of SEQ ID NO: 5, and

(6) a 3′ ITR of SEQ ID NO: 8,

with an optional linker between (1) and (2), between (2) and (3), between (3) and (4), between (4) and (5), and/or between (5) and (6).

In certain embodiments, the vector genome comprises, consists essentially of, or consists of SEQ ID NO: 1, or a polynucleotide having a sequence identity of at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% to SEQ ID NO: 1, encoding an RPE65 polypeptide having a sequence identity of at least 95%, 96%, 97%, 98%, or 99% to SEQ ID NO: 3 (e.g., 100% identical to SEQ ID NO: 3).

In certain embodiments, the vector genome has a sequence identity of at least 99% to SEQ ID NO: 1.

In certain embodiments, the vector genome consists of SEQ ID NO: 1.

In certain embodiments, the AAV9 serotype capsid comprises AAV9 VP1, AAV9 VP2, and AAV9 VP3; or VP1, VP2, and VP3 variants independently having a sequence identity of at least 90%, 91%, 92%, 93%, 94%, 95%, 96%, 97%, 98%, or 99% thereto, respectively.

In certain embodiments, the AAV9 serotype capsid comprises AAV9 VP1 (SEQ ID NO: 9), AAV9 VP2, and AAV9 VP3.

Another aspect of the disclosure provides a recombinant AAV (rAAV) vector genome comprising, consisting essentially of, or consisting of SEQ ID NO: 1, or a polynucleotide having a sequence identity of at least 90%, 91%, 92%, 93%, 94%, 95%, 96%, 97%, 98%, 99%, 99.5%, 99.6%, 99.7%, 99.8%, or 99.9% to SEQ ID NO: 1, encoding an RPE65 polypeptide having a sequence identity of at least 80%, 85%, 90%, 95%, 96%, 97%, 98%, or 99% to SEQ ID NO: 3.

In certain embodiments, the rAAV vector genome is SEQ ID NO: 1, or the polynucleotide having a sequence identity of at least 95% or 99% thereto.

In certain embodiments, the rAAV vector genome is SEQ ID NO: 1.

Another aspect of the disclosure provides a recombinant (rAAV) viral particle has the rAAV vector genome of the disclosure.

In certain embodiments, the rAAV viral particle has a capsid with a serotype of AAV9.

Another aspect of the disclosure provides a recombinant AAV (rAAV) viral particle comprising the rAAV vector genome of the disclosure, encapsidated in a capsid with a serotype of AAV9.

In certain embodiments, the rAAV vector genome is SEQ ID NO: 1.

Another aspect of the disclosure provides a pharmaceutical composition comprising the rAAV vector genome of the disclosure, or the rAAV viral particle of the disclosure, and a pharmaceutically acceptable excipient.

In certain embodiments, the rAAV vector genome is SEQ ID NO: 1.

Another aspect of the disclosure provides a method of treating a RPE65-associated eye disease or disorder (e.g., (human) RPE65-deficient) in a subject in need thereof, comprising administering to the subject a therapeutically effective amount of the rAAV vector genome of the disclosure, the rAAV viral particle of the disclosure, or the pharmaceutically composition of the disclosure, wherein the rAAV vector genome or the rAAV viral particle specifically induces expression of the RPE65 polypeptide from the vector genome of the rAAV viral particle (e.g., in retinal pigment epithelial (RPE) cells).

In certain embodiments, the administrating comprises contacting a cell with the therapeutically effective amount of the rAAV vector genome of the disclosure, the rAAV viral particle of the disclosure, or the pharmaceutically composition of the disclosure.

In certain embodiments, the cell is located in the eye of the subject.

In certain embodiments, the RPE65-associated eye disease or disorder is choroiditis, retinitis pigmentosa, maculopathies, Leber's congenital amaurosis (LCA) including Leber's congenital amaurosis 2 (LCA2), Leber's hereditary optic neuropathy, early onset severe retinal dystrophy, achromatopsia, retinoschisis, ocular albinism, oculocutaneous albinism, Stargardt's disease, Choroideremia, Spinocerebellar Ataxia Type 7 (SCAT), color blindness, lysosomal storage diseases that affect the cornea, such as Mucopolysaccharidosis (MPS) IV and MPS VII, amoebic keratitis, fungal keratitis, bacterial keratitis, viral keratitis, onchocercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, glaucoma, open angle glaucoma, Fundus Flavimaculatus, closed angle glaucoma, pigmentary glaucoma, or a combination thereof. In certain embodiments, the RPE65-associated eye disease or disorder is Leber's congenital amaurosis 2 (LCA2).

In certain embodiments, the subject is a human, such as a human with viable retinal cells.

In certain embodiments, the expression of the RPE65 polypeptide in the cell is increased in comparison to a cell having not been contacted with the rAAV vector genome of the disclosure, the rAAV viral particle of the disclosure, or the pharmaceutically composition of the disclosure.

In certain embodiments, the electroretinogram b-wave amplitude is increased in the eye of the subject by at least about 30%, about 40%, about 50%, about 55%, about 60%, about 65%, about 70%, about 75%, or about 80% compared to the electroretinogram b-wave amplitude prior to the administrating.

In certain embodiments, the increase of the electroretinogram b-wave amplitude in the eye of the subject is stable for at least about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, or about 20 weeks.

In certain embodiments, the rAAV vector genome, the rAAV viral particle, or the pharmaceutical composition is administered via subretinal injection.

In certain embodiments, the subretinal injection is performed following a vitrectomy.

It should be understood that any one embodiment described herein, including those described only in the examples or the claims, can be combined with any one or more additional embodiments of the disclosure unless expressly disclaimed or improper.

### BRIEF DESCRIPTION OF THE DRAWINGS

FIG. 1 is a schematic (not to scale) of an exemplary AAV viral vector genome comprising a (human) RPE65 (retinal pigment epithelium 65) coding sequence.

FIG. 2A shows RPE65 staining in the eye from wild type mouse and knock out RPE65^{-/-} mice untreated and treated with an AAV9 serotype vector AAV9-CAG-RPE65 (“HG-004”). ONL: outer nuclear layer; INL: inner nuclear layer.

FIG. 2B shows visual function measured by electroretinography (ERG) in wild type mouse and knock out RPE65^{-/-} mice untreated and treated with AAV9-CAG-RPE65 (“HG-004”).

FIG. 2C shows a bar graph of electroretinography (Scotopic 3.0 ERG) of the eye from wild type mouse and knock out RPE65^{-/-} mice. Scotopic 3.0 ERG were disrupted in RPE RPE65^{-/-} mice. WT, N=12; RPE65^{-/-}, N=10.

FIG. 3A shows a graph of electroretinography of the eye from knock out RPE65^{-/-} mice treated with AAV9-CAG-hRPE65 at various doses. AAV9-CAG-hRPE65 restored visual function of RPE65^{-/-} mice in a dose dependent manner—visual function was restored to about 50/60/70/72% in RPE65^{-/-} mice at 3, 6, 9, and 14 weeks post HG-004 (3E+7 or Dose 3) dosing. N=3-5 per group. *, P<0.05; **, P<0.01; ***, P<0.001.

FIG. 3B shows a graph of ERG of the eye in knock out RPE65^{-/-} mice treated with AAV9-CAG-hRPE65 post treatment measured at 3, 6, 9, and 14 weeks from FIG. 3A. Dose 1: 3E+6; Dose 3: 3E+7; Dose 4: 1E+8.

FIG. 4 shows a graph of electroretinography of the eye from knock out RPE65^{-/-} mice treated with AAV9-CAG-hRPE65 or AAV2-CAG-hRPE65. AAV9-CAG-hRPE65 (AAV9 capsid) exhibited a better therapeutic effect at much (about 10-100 fold) lower dose compared to the otherwise identical vector genome in AAV2 capsid. N=2-5 per group.

### DETAILED DESCRIPTION

### 1. Overview

The disclosure described herein provides a recombinant AAV (rAAV) viral particle (rAAV) comprising a AAV9 serotype capsid shell (e.g., an AAV9 capsid shell), and an rAAV vector genome comprising a coding sequence encoding a polypeptide, such as RPE65, for the treatment of an eye disease or disorder, such as Leber's congenital amaurosis (LCA).

The disclosure is partly based on the surprising discovery that, an rAAV vector genome comprising a gene sequence encoding a polypeptide, such as RPE65, when encapsidated in an AAV9 capsid shell, provides greater therapeutic effect at much (about 10-100-fold) lower dose than the same vector genome encapsidated in an AAV2 capsid shell.

As used herein, the rAAV vectors, vector genomes, and recombinant AAV viral particles, are referred to herein as rAAV vectors (recombinant adeno-associated virus), vector genomes (e.g., ITR-to-ITR sequences), and recombinant rAAV viral (rAAV) particles, respectively. Specifically, on the one hand, the subject AAV9 serotype rAAV vectors can be composed of any of the same capsid shells found in wild-type AAV9, or variants thereof with similar or identical serotype and tropism (e.g., AAV9 serotype), carrying the rAAV vector genome of the disclosure as viral genetic material. Thus, the subject rAAV vectors possess all the usual advantages derived from the AAV9 shell, such as specific/broad tropism and low immunogenicity.

The rAAV viral vector of the disclosure can be used to deliver the rAAV vector genome comprising a transgene, such as RPE65 (e.g., human RPE65), to a host cell compatible with the tropism of the AAV9 viral capsid shell. The host cell can be from an eye or eye tissue. The eye or eye tissue can be from a mammal, specifically, from a human, such as a human with viable retinal pigment cells.

Thus, one aspect of the disclosure provides a rAAV viral particle comprising an AAV9 serotype capsid, and an rAAV vector genome comprising a transgene encoding an RPE65 gene and used to treat an eye disease or disorder.

In some embodiments, the eye disease or disorder is caused by a genetic mutation. In some embodiments, the genetic mutation is in the RPE65 gene. In some embodiments, the eye disease or disorder is fundus albipunctatusor, choroiditis, retinitis pigmentosa, maculopathies, Leber's congenital amaurosis (LCA) including Leber's congenital amaurosis 2 (LCA2), Leber's hereditary optic neuropathy, early onset severe retinal dystrophy, achromatopsia, retinoschisis, ocular albinism, oculocutaneous albinism, Stargardt's disease, Choroideremia, Spinocerebellar Ataxia Type 7 (SCAT), color blindness, lysosomal storage diseases that affect the cornea, such as Mucopolysaccharidosis (MPS) IV and MPS VII, amoebic keratitis, fungal keratitis, bacterial keratitis, viral keratitis, onchocercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, glaucoma, open angle glaucoma, Fundus Flavimaculatus, closed angle glaucoma, pigmentary glaucoma, or a combination thereof.

In some embodiments, the eye disease or disorder is retinitis pigmentosa, Stargardt's disease, choroiditis, fundus albipunctatusor, or Leber's congenital amaurosis (LCA). In some embodiments, the eye disease or disorder is Leber's congenital amaurosis 2 (LCA2).

Another aspect of the disclosure provides a polynucleotide sequence capable of being packaged into an AAV viral particle, said polynucleotide sequence comprises (not necessarily in this order): (1) a payload or transgene (e.g., an RPE65 coding sequence, including a codon-optimized RPE65 coding sequence optimized for RPE expression in a mammal such as human); (2) an AAV inverted terminal repeat (ITR); (3) a promoter; (4) an optional Kozak sequence; and (5) a polyA sequence.

For example, in a specific embodiment, the vector genome comprises: a) a 5′ inverted terminal repeat (ITR, such as a wild-type AAV2 or AAV9 5′ ITR that can either be flip or flop configuration); b) an RPE65 polynucleotide encoding an RPE65 polypeptide, wherein the RPE65 polynucleotide is at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO: 2; b) a promoter operably linked to and drives the transcription of the RPE65 polynucleotide; c) an optional Kozak sequence; e) a polyA signal sequence; and f) a 3′ ITR (such as a wild-type AAV2 3′ ITR that can either be flip or flop configuration), optionally, wherein the RPE65 polypeptide has the amino acid sequence of SEQ ID NO: 3. In some embodiments, the Kozak sequence is upstream of the RPE65 polynucleotide and downstream of the promoter.

In certain embodiments, the AAV viral particle is derived from AAV9 or with an AAV9 serotype, such as wild-type AAV9.

In certain embodiments, the 5′ ITR and 3′ ITR are derived from AAV2 or AAV9, optionally, the 5′ ITR comprises the nucleotide sequence of SEQ ID NO: 7, and/or and the 3′ ITR comprises the nucleotide sequence of SEQ ID NO: 8.

The VP1 capsid sequence of the wild-type AAV9 is provided in SEQ ID NO: 9.

As used herein, “AAV viral particle” includes viral particles comprising any wild-type capsids of adeno-associated virus (AAV) (belonging to the genus Dependoparvovirus, which in turn belongs to the family Parvoviridae), as well as engineered or variants thereof having modified sequence and/or tissue or host tropism.

As used herein, “AAV9 serotype” includes any AAV9 variants with identical or similar tropism as wild-type AAV9, including all naturally occurring Clad F capsids including AAV0, AAVhu.31, and AAVhu.32. It also includes any improved variants of AAV9 with mutations that does not lead to substantial loss of wt AAV9 tropism (e.g., may include additional functionality not present in wt AAV9), as well as AAV9 mutants with at least 90%,91%,92%,93%,94%,95%,96%,97%,98%,99%,99.2%,99.4%, 99.6%, 99.8%, 99.9% amino acid sequence identity wherein the mutations or sequence changes do not lead to substantial loss of wt AAV9 tropism.

As used herein, “a gene of interest” or “GOI” includes any coding sequence for a protein or polypeptide, including intron and exon sequences, and/or coding sequence for any non-translated RNA or non-coding RNA (ncRNA, such as siRNA, piRNA, short hairpin RNA or shRNA, microRNA or miRNA or precursors thereof including pre-miRNA and pri-miRNA, antisense sequence or oligonucleotide (ASO), guide RNA or gRNA for CRISPR/Cas, rRNA, tRNA, snoRNA, snRNA, exRNA, scaRNA, lncRNA, Xist, and HOTAIR, etc.). A GOI can comprise one coding sequence, or more than one (e.g., 2, 3, 4, 5, 6, 7, 8, 9, 10 or more) coding sequences. The length of the coding sequence of the GOI, or the combined length of all coding sequences of the GOI, is no more than the maximum length of RNA that can be packaged into a particular or chosen AAV viral particle, which can differ from one specific AAV viral particle from another. In certain embodiments, the GOI is RPE65, such as human PRE65 (see, for example, human RPE65 with the nucleotide sequence of SEQ ID NO: 2, or an RPE65 coding sequence encoding the amino acid sequence of SEQ ID NO: 3).

As used herein, “polyA sequence” or “polyA tail” refers to a string of adenine ribonucleotides or adenosine monophosphates (e.g., a string of RNA with each base therewithin an adenine). Such a polyA tail is important for the nuclear export, translation and stability of mRNA. The length of the polyA sequence can vary in different mRNA or the RNA sequence of the disclosure, and can be about 250 nucleotides of polyA, about 230 nucleotides of polyA, about 200 nucleotides of polyA, about 180 nucleotides of polyA, about 160 nucleotides of polyA, about 140 nucleotides of polyA, about 120 nucleotides of polyA, about 100 nucleotides of polyA, or less.

As used herein, “polyA signal sequence” refers to an RNA sequence (such as AAUAAA) that is located downstream of the most 3′ exon and is recognized by an RNA cleavage complex that cleaves off the 3′ terminal sequence of a newly transcribed RNA by RNA polymerase (such as Pol II) such that polyadenylation can occur. Polyadenylate polymerase then adds and extends the poly(A) tail by adding adenosine monophosphate units from ATP to the nascent cleaved 3′ end of the RNA. The initial RNA cleavage is typically catalyzed by the enzyme CPSF (cleavage/polyadenylation specificity factor) and occurs about 10-30 nucleotides downstream of its binding site—the polyA signal sequence, which is often AAUAAA on the transcribed RNA. The sequence at/or immediately 5′ to the site of RNA cleavage is frequently (but not always) CA. The polyA signal sequence recognized by the RNA cleavage complex varies between different groups of eukaryotes, with most human polyadenylation sites containing the AAUAAA sequence, though this sequence is less common in plants and fungi mRNA. In addition, other variants that bind more weakly to CPSF exist. All such sequence motifs recognized by the RNA cleavage complex to enable RNA cleavage and the subsequent polyadenylation are within the scope of the polyA signal sequence.

In some embodiments, the polyA sequence is bovine growth hormone polyadenylation signal (bGH polyA), a small polyA signal (SPA), a human growth hormone polyadenylation signal (hGH polyA), a SV40 polyA signal (SV40 polyA), a rabbit beta globin polyA sequence (rBG polyA), or a variant thereof.

In certain embodiments, the polynucleotide of the disclosure is codon-optimized for expression in a eukaryote, a mammal (such as a human or a non-human mammal), such as in a human eye, especially in human retinal pigment epithelial (RPE) cells.

In a related aspect, the disclosure provides a polynucleotide having (i) one or more (e.g., 1, 2, 3, 4, 5, 6, 7, 8, 9 or 10) nucleotides additions, deletions, or substitutions compared to the subject polynucleotide described above (e.g., SEQ ID NO: 1 or 2); (ii) at least 50%, 60%, 70%, 80%, 90%, 95%, 96%, 97%, 98%, or 99% sequence identity to the subject polynucleotide described above (e.g., SEQ ID NO: 1 or 2); (iii) hybridize under stringent conditions with the subject polynucleotide described above or any of (i) and (ii); or (iv) is a complement of any of (i)-(iii).

In certain embodiments, the polynucleotide is operably linked to a promoter and optionally an enhancer. For example, in some embodiments, the promoter is a constitutive promoter, an inducible promoter, a ubiquitous promoter, or a tissue specific promoter.

In another related aspect, the disclosure provides a vector comprising or encompassing any one of the polynucleotide of the disclosure described herein. The vector can be a cloning vector, or an expression vector. The vector can be a plasmid, phagemid, or cosmid, just to name a few. In certain embodiments, the vector can be used to express the polynucleotide in a mammalian cell, such as a human cell, RPE65 polypeptide of SEQ ID NO: 3, or orthologs, homologs, derivatives, functional fragments, fusions thereof; or any of the polynucleotide of the disclosure; or any of the complex of the disclosure.

In certain embodiments, the vector is a plasmid. In certain embodiments, the vector is a retroviral vector, a phage vector, an adenoviral vector, a herpes simplex viral (HSV) vector, an AAV vector, or a lentiviral vector.

In certain embodiments, the AAV vector is a recombinant AAV vector of the serotype AAV1, AAV2, AAV4, AAV5, AAV6, AAV7, AAVrh74, AAV8, AAV9, AAV10, AAV 11, AAV 12, AAV 13, AAV.PHP.eB, or AAV-DJ.

Another aspect of the disclosure provides a delivery system comprising (1) a delivery vehicle, and (2) the polynucleotide of the disclosure, or the vector of the disclosure.

In certain embodiments, the delivery vehicle is a nanoparticle, a liposome, an exosome, a microvesicle, or a gene-gun.

A further aspect of the disclosure provides a cell or a progeny thereof, comprising the polynucleotide of the disclosure, the vector of the disclosure, the rAAV vector genome of the disclosure, or the rAAV viral particle of the disclosure. The cell can be a prokaryote such as E. coli, or a cell from a eukaryote such as yeast, insect, plant, animal (e.g., mammal including human and mouse). The cell can be isolated primary cell, or established cell lines such as tumor cell lines, 293 cells or 293T cells, or stem cells, iPCs, etc. In certain embodiments, the host cell is a mammalian cell, such as a HEK293 cell or a variant thereof (e.g., HEK293T cell), a Vero cell or variant thereof, or an insect cell, such as Sf9 or Sf21 cells.

Another aspect of the disclosure provides a method to produce the AAV viral particle of the disclosure, comprising packaging the vector genome of the disclosure in a suitable packaging cell line to encapsidate the vector genome inside an AAV capsid of AAV9 serotype. In certain embodiments, the method further comprises isolating or purifying the rAAV viral particles of the disclosure.

In certain embodiments, the cell or progeny thereof is a eukaryotic cell (e.g., a non-human mammalian cell, a human cell, or a plant cell) or a prokaryotic cell (e.g., a bacteria cell).

In certain embodiments, the AAV viral particle of the disclosure is produced in a mammalian cell, such as HeLa cell or HEK293/293T cells, by triple transfection of a plasmid encoding AAV helper function, a plasmid comprising a GOI (e.g., RPE65) flanked by ITR sequences (e.g., AAV2 or AAV9) for AAV packaging, and a plasmid comprising rep/cap coding sequences.

In certain embodiments, the AAV viral particle of the disclosure is produced in an insect cell (e.g., Sf9) through the baculoviral packaging system.

In certain embodiments, the AAV viral particle of the disclosure is produced in an HSV compatible packaging system comprising Vero cells and ICP27-deleted HSV vectors.

A further aspect of the disclosure provides a non-human multicellular eukaryote comprising the cell, the vector genome, or the viral particle of the disclosure.

In certain embodiments, the non-human multicellular eukaryote is an animal (e.g., rodent or primate, e.g., non-human primate (NHP)) model for a human genetic disorder. In certain embodiments, the NHP is a monkey, such as Cynomolgus Monkeys (Macaca fascicularis).

In certain embodiments, the ITR sequence is at the 3′ end and the 5′ end. In certain embodiments, the 3′ ITR and the 5′ ITR are derived from AAV2 or AAV9, such as wt AAV2 ITR sequences.

In a related aspect, the disclosure provides a eukaryotic cell comprising a rAAV viral genome, said rAAV viral genome comprising: (1) a nucleic acid molecule encoding a gene of interest, e.g., an RPE65 polypeptide or variant thereof; (2) a promoter operably linked to the nucleic acid molecule; and (3) a 3′ ITR and a 5′ ITR. In some embodiments, the rAAV viral genome further comprises a Kozak sequence and a polyA sequence.

In another aspect, the disclosure provides a composition comprising one or more vectors of the disclosure, said one or more vectors comprise: (i) a first polynucleotide that encodes RPE65, such as a RPE65 proteins based on SEQ ID NO: 3, or orthologs, homologs, derivatives, functional fragments, fusions thereof, optionally operably linked to a first regulatory element and a second regulatory element; and (ii) a polyA sequence. The first and the second polynucleotides are on the same vector. In some embodiments, the first regulatory element is a promoter, such as an inducible promoter or tissue-specific promoter. In some embodiments, the second regulatory element is a Kozak sequence.

In some embodiments, the vector is a plasmid. In some embodiments, the vector is a viral vector based on a retrovirus, a replication incompetent retrovirus, adenovirus, replication incompetent adenovirus, or AAV. In some embodiments, the vector can self-replicate in a host cell (e.g., having a bacterial replication origin sequence). In some embodiments, the vector can integrate into a host genome and be replicated therewith. In some embodiments, the vector is a cloning vector. In some embodiments, the vector is an expression vector.

The disclosure further provides a delivery composition for delivering the RPE65 polypeptide based on SEQ ID NOs: 2 or 3, or orthologs, homologs, derivatives, conjugates, functional fragments, fusions thereof of the disclosure; the polynucleotide of the disclosure; the vector of the disclosure; the cell of the disclosure, and the composition of the disclosure. The delivery can be through any way known in the art, such as transfection, lipofection, electroporation, gene gun, microinjection, sonication, calcium phosphate transfection, cation transfection, viral vector delivery, etc., using vehicles such as liposome(s), nanoparticle(s), exosome(s), microvesicle(s), a gene-gun or one or more viral vector(s).

The disclosure further provides a kit comprising any one or more of the following: the RPE65 polypeptide of the disclosure based on SEQ ID NOs: 2 or 3, or orthologs, homologs, derivatives, conjugates, functional fragments, fusions thereof of the disclosure; the polynucleotide of the disclosure; the vector of the disclosure; the rAAV vector genome of the disclosure; the rAAV viral particle of the disclosure, the cell of the disclosure, and the composition of the disclosure. In some embodiments, the kit may further comprise an instruction for how to use the kit components, and/or how to obtain additional components from 3^{rd} party for use with the kit components. Any component of the kit can be stored in any suitable container.

A further aspect of the disclosure provides a method of delivering a gene of interest (GOI) into a cell or an animal, comprising contacting the cell or the animal with the rAAV viral particle of the disclosure, or the population of the rAAV viral particles of the disclosure, wherein the GOI is encoded by the polynucleotide sequence of the disclosure.

With the disclosures generally described herein above, more detailed descriptions for the various aspects of the disclosure are provided in separate sections below. However, it should be understood that, for simplicity and to reduce redundancy, certain embodiments of the disclosure are only described under one section or only described in the claims or examples. Thus, it should also be understood that any one embodiment of the disclosure, including those described only under one aspect, section, or only in the claims or examples, can be combined with any other embodiment of the disclosure, unless specifically disclaimed or the combination is improper.

### 2. Polynucleotides or Nucleic Acids

The disclosure also provides polynucleotides or nucleic acids encoding the proteins described herein (e.g., an RPE65 protein).

In some embodiments, the nucleic acid is a synthetic nucleic acid. In some embodiments, the nucleic acid is a DNA molecule. In some embodiments, the nucleic acid is an RNA molecule (e.g., an mRNA molecule encoding an RPE65 polypeptide). In some embodiments, the mRNA is capped, polyadenylated, substituted with 5-methyl cytidine, substituted with pseudouridine, or a combination thereof.

In certain embodiments, the polynucleotide or nucleic acid encoding the proteins described herein (e.g., an RPE65 protein) comprises or is operably linked to a transcriptional regulatory sequence, such as a promoter, that drive the transcription of the polynucleotide.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence. In other instances, this sequence may also include an enhancer sequence and other regulatory elements which are required for expression of the gene product. The promoter/regulatory sequence may, for example, be one which expresses the gene product (e.g., the RNA sequence of the disclosure) in a tissue or cell type specific manner.

As used herein, the term “operable linkage” or “operably linked” refers to a physical or functional juxtaposition of the components so described as to permit them to function in their intended manner. In the example of an expression control element in operable linkage with a heterologous polynucleotide (e.g., an RPE65 coding sequence), the relationship is such that the control element modulates expression of the heterologous polynucleotide. More specifically, for example, two DNA sequences operably linked means that the two DNAs are arranged (cis or trans) in such a relationship that at least one of the DNA sequences is able to exert a physiological effect upon the other sequence.

In some embodiments, the nucleic acid (e.g., DNA) is operably linked to a regulatory element (e.g., a promoter) in order to control the expression of the nucleic acid (e.g., an RPE65 nucleic acid). In some embodiments, the promoter is ubiquitous. In some embodiments, the promoter is a constitutive promoter. In some embodiments, the promoter is an inducible promoter. In some embodiments, the promoter is a cell-specific promoter. In some embodiments, the promoter is an organism-specific promoter, e.g., tissue-specific promoter.

In certain embodiments, the promoter is a constitutive promoter.

As used herein, a “constitutive” promoter is a nucleotide sequence which, when operably linked with a polynucleotide which encodes or specifies a gene product, causes the gene product to be produced in a cell under most or all physiological conditions of the cell.

In certain embodiments, the promoter is an inducible promoter.

As used herein, an “inducible” promoter is a nucleotide sequence which, when operably linked with a polynucleotide which encodes or specifies a gene product, causes the gene product to be produced in a cell substantially only when an inducer which corresponds to the promoter is present in the cell.

In certain embodiments, the promoter is a tissue-specific promoter, a species-specific promoter, or a cell cycle-specific promoter.

As used herein, a “tissue- or cell-type-specific” promoter is a nucleotide sequence which, when operably linked with a polynucleotide encodes or specified by a gene, causes the gene product to be produced in a specific cell type or a specific tissue preferentially, due to, for example, the cell/tissue is a cell type or tissue type in which the promoter is normally active.

In some embodiments, the promoter is heterologous to the gene of interest. In some embodiments, the promoter is the natural promoter of the gene of interest. In some embodiments, the heterologous promoter includes an insertion, deletion, substitution, and/or other mutation. In some embodiments, the natural promoter includes an insertion, deletion, substitution, and/or other mutation.

In certain embodiments, the promoter is a Pol II promoter. In certain embodiments, the promoter is a Pol III promoter, such as U6 promoter.

Suitable promoters are known in the art and include, for example, a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (IE) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nP2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, or a functional fragment thereof.

In some embodiments, the promoter is a CAG promoter (e.g., SEQ ID NO: 4), or a variant thereof, which can be used to regulate the expression of the RPE65 coding sequence described herein.

In some embodiments, the nucleic acid(s) are present in a vector (e.g., a viral vector or a phage). The vector can be a cloning vector, or an expression vector. The vectors can be plasmids, phagemids, cosmids, etc. The vectors may include one or more regulatory elements that allow for the propagation of the vector in a cell of interest (e.g., a bacterial cell or a mammalian cell). In some embodiments, the vector includes a nucleic acid encoding a gene of interest, e.g., RPE65, as described herein. In some embodiments, the vector includes multiple nucleic acids, each encoding a gene of interest, or multiple copies of one gene of interest, e.g., multiple copies of RPE65, as described herein.

In one aspect, the present disclosure provides nucleic acid sequences that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, i.e., nucleic acid sequences encoding a gene of interest, e.g., RPE65.

In another aspect, the present disclosure also provides nucleic acid sequences encoding amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences of the subject RPE65 polypeptide.

In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is the same as the sequences described herein. In some embodiments, the nucleic acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 nucleotides, e.g., contiguous or non-contiguous nucleotides) that is different from the sequences described herein.

In related embodiments, the disclosure provides amino acid sequences having at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is the same as the sequences described herein. In some embodiments, the amino acid sequences have at least a portion (e.g., at least 1, 2, 3, 4, 5, 6, 7, 8, 9, 10, 11, 12, 13, 14, 15, 20, 30, 40, 50, 60, 70, 80, 90, or 100 amino acid residues, e.g., contiguous or non-contiguous amino acid residues) that is different from the sequences described herein.

To determine the percent sequence identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blosum 62 scoring matrix with a gap penalty of 12, a gap extend penalty of 4, and a frameshift gap penalty of 5.

The proteins described herein (e.g., an RPE65 polypeptide) can be delivered or used as either nucleic acid molecules or polypeptides.

In certain embodiments, the nucleic acid molecule encoding the RPE65 polypeptide, derivatives or functional fragments thereof are codon-optimized for expression in a host cell or organism. The host cell may include established cell lines (such as 293T cells) or isolated primary cells. The nucleic acid can be codon optimized for use in any organism of interest, in particular human cells or bacteria. For example, the nucleic acid can be codon-optimized for any prokaryotes (such as E. coli), or any eukaryotes such as human and other non-human eukaryotes including yeast, worm, insect, plants and algae (including food crop, rice, corn, vegetables, fruits, trees, grasses), vertebrate, fish, non-human mammal (e.g., mice, rats, rabbits, dogs, birds (such as chicken), livestock (cow or cattle, pig, horse, sheep, goat etc.), or non-human primates). Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference in its entirety). Computer algorithms for codon optimizing a particular sequence for expression in a particular host cell are also available, such as Gene Forge (Aptagen; Jacobus, Pa.).

An example of a codon optimized sequence is in this instance a sequence optimized for expression in a eukaryote, e.g., humans (i.e., being optimized for expression in humans), or for another eukaryote, animal or mammal as herein discussed. Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at http://www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a RPE65 polypeptide correspond to the most frequently used codon for a particular amino acid.

### 3. Vectors (Plasmids or Bacmids)

As used herein, a “vector” generally refers to a composition of matter which comprises an isolated nucleic acid and which can be used to deliver the isolated nucleic acid to the interior of a cell.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids, bacmids (e.g., naked or contained in liposomes) and viruses (e.g., lentiviruses, retroviruses, adenoviruses, and adeno-associated viruses) that incorporate the recombinant polynucleotide.

An rAAV vector genome sequence of the disclosure comprising a GOI, e.g., an RPE65 polypeptide, is a vector for delivering the GOI into a target/host cell through a rAAV viral particle encapsidating the vector genome.

In certain embodiments, the RPE65 polynucleotide sequence of the disclosure encodes amino acid sequences that are at least 50%, 55%, 60%, 65%, 70%, 75%, 80%, 85%, 90%, 91%, 92%, 93%, 94%, 95%, 96%, 97%, 98%, 99%, or 100% identical to the amino acid sequences of the wild-type RPE65.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes should be at least 80% of the length of the reference sequence, and in some embodiments is at least 90%, 95%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blosum 62 scoring matrix with a gap penalty of 12, a gap extend penalty of 4, and a frameshift gap penalty of 5.

In certain embodiments, the nucleic acid molecule encoding the RPE65 protein are codon-optimized for expression in a host cell or organism. The host cell may include established cell lines (such as HeLa, 293, or 293T cells) or isolated primary cells. The nucleic acid can be codon optimized for use in any organism of interest, in particular human cells or bacteria. For example, the nucleic acid can be codon-optimized for any prokaryotes (such as E. coli), or any eukaryotes such as human and other non-human eukaryotes including yeast, worm, insect, plants and algae (including food crop, rice, corn, vegetables, fruits, trees, grasses), vertebrate, fish, non-human mammal (e.g., mice, rats, rabbits, dogs, birds (such as chicken), livestock (cow or cattle, pig, horse, sheep, goat etc.), or non-human primates). Codon usage tables are readily available, for example, at the “Codon Usage Database” available at www.kazusa.orjp/codon/, and these tables can be adapted in a number of ways. See Nakamura et al., Nucl. Acids Res. 28:292, 2000 (incorporated herein by reference in its entirety). Computer algorithms for codon optimizing a particular sequence for expression in a particular host cell are also available, such as Gene Forge (Aptagen; Jacobus, Pa.).

An example of a codon optimized sequence is in this instance a sequence optimized for expression in a eukaryote, e.g., humans (i.e., being optimized for expression in humans), or for another eukaryote, animal or mammal as herein discussed. Whilst this is preferred, it will be appreciated that other examples are possible and codon optimization for a host species other than human, or for codon optimization for specific organs is known. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid.

Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at http://www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a Cas correspond to the most frequently used codon for a particular amino acid.

In certain embodiments, the vector genome comprises a 5′ Inverted Terminal Repeat (ITR) sequence, a 3′ ITR sequence, or both.

Inverted Terminal Repeat (ITR) sequences are important for initiation of viral DNA replication and circularization of adeno-associated virus genomes. Within the ITR sequences, secondary structures (e.g., stems and loops formed by palindromic sequences) are important one or more ITR functions in viral replication and/or packaging. Such sequence elements include the RBE sequence (Rep binding element), RBE′ sequence, and the trs (terminal resolution sequence).

In certain embodiments, the rAAV vector genome comprises a 5′ AAV ITR sequence and a 3′ AAV ITR sequence.

In certain embodiments, the 5′ and the 3′ AAV ITR sequences are both wild-type AAV ITR sequences from AAV1, AAV2, AAV3A, AAV3B, AAV4, AAV5, AAV6, AAV7, AAVrh74, AAV8, AAV9, AAV10, AAV11, AAV12, AAV13, or a member of the Clade to which any of the AAV1-AAV13 belong.

In certain embodiments, the 5′ and the 3′ AAV ITR sequences are both wild-type AAV ITR sequences from AAV2.

In certain embodiments, the 5′ and/or 3′ ITR sequences are modified ITR sequences. For example, the most 5′ end or the most 3′ end of the wild-type ITR sequences (e.g., AAV2 ITR sequences) may be deleted. The deletion can be up to 18, 17, 16, 15, 14, 13, 12, 11, 10, 9, 8, 7, 6, 5, 4, 3, 2, or 1 nucleotide.

In certain embodiments, up to 15 (such as exactly 15) nucleotides of the most 5′ end nucleotides, and/or up to 15 (such as exactly 15) nucleotides of the most 3′ end nucleotides, of the wild-type AAV2 ITR sequences may be deleted.

Thus the 5′ and/or 3′ modified ITR(s) may comprising up to 144, 143, 142, 141, 140, 139, 138, 137, 136, 135, 134, 133, 132, 131, 130, 129, 128, or 127-nt (such as 130 nucleotides) of the 145-nt wild-type AAV ITR sequences.

In certain embodiments, the modified ITR sequences comprise the RBE sequence, the RBE′ sequence, and/or the trs of the wt ITR sequence.

In certain embodiments, the modified ITR sequences comprise both the RBE sequence and the RBE′ sequence.

In certain embodiments, the modified ITR sequences confer stability of the plasmids of the disclosure comprising the AAV vector genome (see below) in bacteria, such as stability during plasmid production.

In certain embodiments, the modified ITRs do not interfere with sequencing verification of the plasmids of the disclosure comprising the AAV vector genome.

In certain embodiments, the modified 5′ ITR sequence comprises a 5′ heterologous sequence that is not part of wild-type AAV 5′ ITR sequence. In certain embodiments, the modified 3′ ITR sequence comprises a 3′ heterologous sequence that is not part of wild-type AAV 3′ ITR sequence.

In certain embodiments, the modified 5′ ITR sequence comprises a 5′ heterologous sequence that is not part of wild-type AAV (e.g., wt AAV2) 5′ ITR sequence, and the modified 3′ ITR sequence comprises a 3′ heterologous sequence that is not part of wild-type AAV (e.g., wt AAV2) 3′ ITR sequence, wherein the 5′ heterologous sequence and the 3′ heterologous sequence are complementary to each other.

In certain embodiments, the 5′ heterologous sequence and the 3′ heterologous sequence each comprises a type II restriction endonuclease recognition sequence, such as recognition sequence for Sse8387I (CCTGCAGG), or recognition sequence for Pac (TTAATTAA).

In certain embodiments, the 5′ heterologous sequence comprises, consists essentially of, or consists of CCTGCAGGCAG (SEQ ID NO: 11), and the 3′ heterologous sequence comprises, consists essentially of, or consists of the reverse complement of SEQ ID NO: 11.

In certain embodiments, the 5′ heterologous sequence comprises, consists essentially of, or consists of TTAATTAAGG (SEQ ID NO: 12), and the 3′ heterologous sequence comprises, consists essentially of, or consists of the reverse complement of SEQ ID NO: 12.

In certain embodiments, the 5′ ITR and the 3′ ITR are both flip ITR's.

In certain embodiments, the 5′ ITR and the 3′ ITR are both flop ITR's.

In certain embodiments, the 5′ ITR and the 3′ ITR are independently flip or flop ITR's.

In certain embodiments, the 5′ ITR is a flip ITR, and the 3′ ITR is a flop ITR.

In certain embodiments, the 5′ ITR is a flop ITR, and the 3′ ITR is a flip ITR.

In certain embodiments, the 5′ ITR is a flip ITR, and the 3′ ITR is a flip ITR.

In certain embodiments, the 5′ ITR is a flop ITR, and the 3′ ITR is a flop ITR.

As used herein, a 5′ flip ITR has the B:B′ segment closer to the 5′-terminal than the C:C′ segment. A 3′ flip ITR has the B:B′ segment closer to the 3′-terminal than the C:C′ segment. A 5′ flop ITR has the C:C′ segment closer to the 5′-terminal than the B:B′ segment. A3′ flop ITR has the C:C′ segment closer to the 3′-terminal than the B:B′ segment.

In certain embodiments, the modified 5′ ITR and the modified 3′ ITR are both flop ITRs, the modified 5′ ITR comprises a 5′ heterologous sequence that is not part of wild-type AAV2 5′ ITR sequence (such as SEQ ID NO: 11 or 12), and the modified 3′ ITR sequence comprises a 3′ heterologous sequence that is not part of wild-type AAV2 3′ ITR sequence, wherein the 5′ heterologous sequence and the 3′ heterologous sequence are complementary to each other, and each comprises a type II restriction endonuclease recognition sequence, such as recognition sequence for Sse8387I or PacI; optionally, said modified 5′ ITR sequence further comprises a deletion in the C:C′ segment, such as an 11-nts deletion AAAGCCCGGGC (SEQ ID NO: 13).

In certain embodiments, the 5′ ITR comprises up to 141 nt of the most 3′ nucleotides of the 145-nt wt AAV2 5′ ITR (e.g., a deletion of 4 or more most 5′ end of the 145-nt wt AAV2 5′ ITR).

In certain embodiments, the 5′ ITR comprises up to 130 nt of the most 3′ nucleotides of the 145-nt wt AAV2 5′ ITR (e.g., a deletion of 15 or more most 5′ end of the 145-nt wt AAV2 5′ ITR).

In certain embodiments, the 3′ ITR comprises up to 141 nt of the most 5′ nucleotides of the 145-nt wt AAV2 3′ ITR (e.g., a deletion of 4 or more most 3′ end of the 145-nt wt AAV2 3′ ITR).

In certain embodiments, the 3′ ITR comprises up to 130 nt of the most 5′ nucleotides of the 145-nt wt AAV2 3′ ITR (e.g., a deletion of 15 or more most 3′ end of the 145-nt wt AAV2 3′ ITR).

In certain embodiments, the 5′ and 3′ ITR sequences are compatible for AAV production in mammalian-cell based on triple transfection.

In certain embodiments, the 5′ and 3′ ITR sequences are compatible for AAV production in insect cell (e.g., Sf9) based on baculovirus vector (see below).

In certain embodiments, the 5′ and 3′ ITR sequences are compatible for AAV production in mammalian-cell based on HSV vectors (see below).

In certain embodiments, the rAAV vector genome of the disclosure further comprises a Kozak sequence or a functional variant thereof. In certain embodiments, the Kozak sequence is SEQ ID NO: 6; or a sequence comprising at most 1, 2, 3, or 4 nucleotide differences from SEQ ID NO: 6, and optionally wherein the last three nucleotide is ACC or GCC.

In certain embodiments, the rAAV vector genome of the disclosure further comprises a polyadenylation (polyA) signal sequence. In certain embodiments, the polyA signal sequence is selected from the group consisting of growth hormone polyadenylation signal (bGH polyA), a small polyA signal (SPA), a human growth hormone polyadenylation signal (hGH polyA), a SV40 polyA signal (SV40 polyA), a rabbit beta globin polyA signal (rBG polyA), or a variant thereof. In certain embodiments, the polyA signal sequence is SV40 polyA signal sequence or a functional variant thereof.

In certain embodiments, the rAAV vector genome is SEQ ID NO: 1, or the polynucleotide at least 95% or 99% identical thereto. In certain embodiments, the rAAV vector genome is SEQ ID NO: 1.

In certain embodiments, the rAAV vector genome that are at least 50%, 55%, 60%, 65%, 70%, 75%, 80%, 85%, 90%, 91%, 92%, 93%, 94%, 95%, 96%, 97%, 98%, 99%, or 100% identical to the nucleic acid sequences described herein, e.g., nucleic acid sequences (such as ITR-to-ITR sequences, for example, SEQ ID NO: 1).

In some embodiments, the rAAV vector genome is present in a vector (e.g., a viral vector or a phage, such as an HSV vector, a baculovirus vector, or an AAV vector). The vector can be a cloning vector, or an expression vector. The vectors can be plasmids, phagemids, Cosmids, etc. The vectors may include one or more regulatory elements that allow for the propagation of the vector in a cell of interest (e.g., a bacterial cell, insect cell, or a mammalian cell).

In certain embodiments, the rAAV vector genome of the disclosure is encoded by a DNA expression vector, such as a plasmid or bacmid (e.g., one that can be maintained or replicated like a baculovirus inside an insect cell). Such DNA expression vector can transcribe the RNA sequence of the disclosure within a suitable host cell, such as a mammalian packaging cell (e.g., HEK293T cells) or an insect packaging cell (e.g., Sf9 cells), such that the subject rAAV viral particles can be produced in the presence of other elements necessary for rAAV packaging (such as rep and cap coding sequences).

Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In some embodiments, the rAAV vector genome is replicated from a plasmid or bacmid. The plasmid or bacmid can include the gene of interest sequence. In some embodiments, the promoter is operably linked to the gene of interest and is located upstream of the gene of interest.

### 4. AAV Particles and Populations of AAV Particles

In certain embodiments, the disclosure provides an isolated rAAV viral particle comprising any one of the polynucleotide sequence of the disclosure encapsidated within an AAV9, or variant thereof, capsid or viral particle described herein.

In certain embodiments, the isolated rAAV viral particle comprises an AAV9 capsid.

In certain embodiments, the isolated rAAV viral particle comprises a Clade F AAV capsids, or mutants/derivatives based on AAV9 (e.g., sharing significant sequence homology and spectrum of tropism as AAV9).

In some embodiments, the AAV capsid or viral particle is of a serotype or a combination of one or more serotypes described herein.

A related aspect of the disclosure also provides a population of isolated rAAV viral particle of the disclosure.

In some embodiments, the population of rAAV viral particles contain a plurality of rAAV viral particles of the disclosure, wherein about 40%, 50%, 60%, 70%, 80%, 90%, 95%, 96%, 97%, 98%, 99% or more of the rAAV particles within the population have encapsidated rAAV vector genome sequence of the disclosure.

### 5. Host Cells and AAV Production

General principles of rAAV production are known in the art. See review in, for example, Carter (Current Opinions in Biotechnology, 1533-539, 1992); and Muzyczka, Curr Topics in Microbial, and Immunol 158:97-129, 1992, both incorporated herein by reference). Various approaches are described in Ratschin et al (Mol. Cell. Biol. 4:2072, 1984; Hermonat et al. (Proc. Natl. Acad. Sci. USA 81:6466, 1984); Tratschin et al. (Mol. Cell. Biol. 5:3251, 1985); McLaughlin et al. (J. Virol 62:1963, 1988); and Lebkowski et al. (Mol. Cell. Biol 7:349, 1988), Samulski et al. (J. Virol 63:3822-3828, 1989); U.S. Pat. No. 5,173,414; WO 95/13365 and U.S. Pat. No. 5,658,776; WO 95/13392; WO 96/17947; PCT/US98/18600; WO 97/09441; WO 97/08298; WO 97/21825; WO 97/06243; WO 99/11764; Perrin et al. (Vaccine 13:1244-1250, 1995; Paul et al. (Human Gene Therapy 4:609-615, 1993); Clark et al. (Gene Therapy 3:1124-1132, 1996; U.S. Pat. Nos. 5,786,211; 5,871,982; and 6,258,595.

AAV vector serotypes can be matched to target cell types. For example, Table 2 of WO2018002719A1 lists exemplary cell types that can be transduced by the indicated AAV serotypes (incorporated herein by reference).

Packaging cells are used to form virus particles that are capable of infecting a host cell. Such cells include HEK293 and Sf9 cells, which can be used to package AAV and adenovirus.

Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions can be supplied in trans by the packaging cell line, usually as a result of expression of these viral functions/proteins (such as the rep and cap genes for AAV) either as transgenes integrated into the packaging cell, or as transgenes on a second viral vector or expression vector introduced into the packaging cell.

For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a gene of interest) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some embodiments, the subject rAAV viral particle is produced using a baculovirus expression system packaged in insect cells such as Sf9 cells. See, for example, WO2007046703, WO2007148971, WO2009014445, WO2009104964, WO2013036118, WO2011112089, WO2016083560, WO2015137802, and WO2019016349, all incorporated herein by reference.

The vector titers are usually expressed as viral genomes per ml (vg/ml). In certain embodiments, viral titers are above 1×10⁹, above 5×10¹⁰, above 1×10¹¹, above 5×10¹¹, above 1×10¹², above 5×10¹², or above 1×10¹³ vg/ml.

### 6. Cells and Therapeutic Applications

One aspect of the disclosure provides an rAAV vector genome or rAAV viral particle comprising the rAAV vector genome, comprising a polynucleotide encoding a gene of interest, e.g., RPE65 homologs, orthologs, fusions, derivative, conjugates, or functional fragments thereof as described herein. In some embodiments, the rAAV vector genome further comprises a promoter, a Kozak sequence, and/or a polyA sequence. In some embodiments, the rAAV viral particle comprises an AAV capsid, e.g., AAV9 capsid. In some embodiments, the AAV serotype is derived from AAV9.

In a related aspect, the disclosure also provides a cell comprising any of the rAAV viral particle or rAAV vector genome of the disclosure. In certain embodiments, the cell is a prokaryote. In certain embodiments, the cell is a eukaryote. In certain embodiments, the cell is mammalian or non-mammalian. In certain embodiments, the cell is human.

The rAAV vector genome and rAAV viral particle described herein can have various therapeutic applications. Such applications may be based on one or more of the abilities below, both in vitro and in vivo, of the rAAV viral particle to deliver a polynucleotide to a cell to express a gene of interest, e.g., an RPE65 polypeptide, to replace a mutated or missing endogenous copy of the RPE65 gene. The term “treatment” or “treating” refers to administration of a composition as disclosed herein (e.g., an AAV comprising a transgene and/or cells) to a subject for purposes including 1) preventing or protecting against the disease or condition, that is, causing the clinical symptoms not to develop; 2) inhibiting the disease or condition, that is, arresting, slowing down, ameliorating or suppressing the development of clinical symptoms; 3) relieving the disease or condition, that is, causing the regression of clinical symptoms; and/or 4) replacing and/or restoring the function loss of the diseased cells, tissue and/or organ. In some embodiments, the term “treatment” or “treating” refers to relieving the disease or condition; that is, causing the regression of clinical symptoms. In some embodiments, the term “treatment” or “treating” alternately or additionally refers to the prophylactic treatment of a subject in need thereof. The prophylactic treatment can be accomplished by providing an appropriate dose of a therapeutic agent to a subject at risk of suffering from an ailment, thereby substantially averting onset of the ailment. It will be understood by those skilled in the art that it is not always possible to distinguish between “preventing” and “suppressing”, since the ultimate inductive event or events may be unknown or latent, or the patient may not be ascertained until well after the occurrence of the event or events. Therefore, as used herein, the term “prophylaxis” is intended as an element of “treatment” to encompass both “preventing” and “suppressing” as defined herein.

The term “subject” refers to an animal, a such as a mammal, e.g., a human. The methods described herein can be useful in human therapeutics, pre-clinical, and veterinary applications. In some embodiments, the subject is a mammal, and in some embodiments, the subject is human.

In certain embodiments, the methods of the disclosure can be used to treat an eye disease or disorder. In some embodiments, the eye disease or disorder is amoebic keratitis, fungal keratitis, bacterial keratitis, viral keratitis, onchocercal keratitis, keratoconjunctivitis, bacterial keratoconjunctivitis, viral keratoconjunctivitis, vernal keratoconjunctivitis, atopic keratoconjunctivitis, corneal dystrophic diseases, Fuchs' endothelial dystrophy, Sjogren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, noninfectious uveitis, anterior uveitis, posterior uveitis (including toxoplasmosis), pan-uveitis, an inflammatory disease of the vitreous or retina, endophthalmitis prophylaxis and treatment, macular edema, macular degeneration, wet age related macular degeneration (wet AMD), dry age related macular degeneration (dry AMD), diabetic macular edema (DME), allergic conjunctivitis, proliferative and non-proliferative diabetic retinopathy, hypertensive retinopathy, an autoimmune disease of the retina, primary and metastatic intraocular melanoma, other intraocular metastatic tumors, open angle glaucoma, Stargardt's disease, Fundus Flavimaculatus, closed angle glaucoma, pigmentary glaucoma, retinitis pigmentosa (RP), Leber's congenital amaurosis (LCA) including Leber's congenital amaurosis 2 (LCA2), Usher's syndrome, Choroideremia, a rod-cone or cone-rod dystrophy, a ciliopathy, a mitochondrial disorder, progressive retinal atrophy, a degenerative retinal disease, geographic atrophy, a familial or acquired maculopathy, a retinal photoreceptor disease, a retinal pigment epithelial-based disease, cystoid macular edema, retinal detachment, traumatic retinal injury, iatrogenic retinal injury, macular holes, macular telangiectasia, a ganglion cell disease, an optic nerve cell disease, optic neuropathy, ischemic retinal disease, retinopathy of prematurity, retinal vascular occlusion, familial macroaneurysm, a retinal vascular disease, an ocular vascular diseases, a vascular disease, an ischemic optic neuropathy disease, diabetic retinal oedema, senile macular degeneration due to sub-retinal neovascularization, myopic retinopathy, retinal ischemia, choroidal vascular insufficiency, choroidal thrombosis and neovascular retinopathies resulting from carotoid artery ischemia, corneal neovascularisation, a corneal disease or opacification with an exudative or inflammatory component, diffuse lamellar keratitis, neovascularisation due to penetration of the eye or contusive ocular injury, rubosis iritis, Fuchs' heterochromic iridocyclitis, chronic uveitis, anterior uveitis, inflammatory conditions resulting from surgeries such as LASIK, LASEK, refractive surgery, IOL implantation; irreversible corneal oedema as a complication of cataract surgery, oedema as a result of insult or trauma, inflammation, infectious and non-infectious conjunctivitis, iridocyclitis, iritis, scleritis, episcleritis, superficial punctuate keratitis, keratoconus, posterior polymorphous dystrophy, Fuch's dystrophies, aphakic and pseudophakic bullous keratopathy, corneal oedema, scleral disease, ocular cicatrcial pemphigoid, pars planitis, Posner Schlossman syndrome, Behcet's disease, Vogt-Koyanagi-Harada syndrome, hypersensitivity reactions, ocular surface disorders, conjunctival oedema, Toxoplasmosis chorioretinitis, inflammatory pseudotumor of the orbit, chemosis, conjunctival venous congestion, periorbiatal cellulits, acute dacroycystitis, non-specific vasculitis, sarcoidosis, cytomegalovirus infection, and combinations thereof. In certain embodiments, the eye disease or disorder is age related macular degeneration. In some embodiments, the eye disease or disorder is LCA, retinitis pigmentosa, Stargardt's disease, or choroiditis. In some embodiments, the eye disease or disorder is LCA. In some embodiments, the eye disease or disorder is Leber's congenital amaurosis 2 (LCA2).

### 7. Delivery

Through this disclosure and the knowledge in the art, the rAAV vector genome and/or rAAV viral particle described herein comprising nucleic acid molecules encoding a gene of interest, e.g., an RPE65 polypeptide or variants thereof, can be delivered by various delivery systems such as vectors, e.g., plasmids and viral delivery vectors, using any suitable means in the art. Such methods include (and are not limited to) electroporation, lipofection, microinjection, transfection, sonication, gene gun, etc.

In certain embodiments, the RPE65 polypeptide or variants can be delivered using suitable vectors, e.g., plasmids or viral vectors, such as adeno-associated viruses (AAV), lentiviruses, adenoviruses, retroviral vectors, and other viral vectors, or combinations thereof. The RPE65 coding sequence can be packaged into one or more vectors, e.g., plasmids or viral vectors. For bacterial applications, the nucleic acids encoding an RPE65 polypeptide, or variant thereof, described herein can be delivered to the bacteria using a phage. Exemplary phages, include, but are not limited to, T4 phage, Mu, λ phage, T5 phage, T7 phage, T3 phage, Φ29, M13, MS2, Qβ, and ΦX174.

In certain embodiments, the delivery is through AAV9 serotype viral vectors, such as AAV9 or other Clade F capsids, or mutants/derivatives based on AAV9 (e.g., sharing significant sequence homology and spectrum of tropism as AAV9).

In some embodiments, the vectors, e.g., plasmids or viral vectors (e.g., AAV viral vectors), are delivered to the tissue of interest by, e.g., intramuscular injection, intravenous administration, transdermal administration, intranasal administration, oral administration, or mucosal administration.

In certain embodiments, the AAV viral particle of the disclosure (e.g., AAV9 viral particle) is delivered through subretinal injection, such as subretinal injection following a vitrectomy. In certain embodiments, the delivery is one subretinal injection per eye. For example, under adequate anesthesia, a subretinal injection of a therapeutically effective amount of the vector genomes (vg) of the disclosure in a suitable total volume (e.g., about 0.1-0.5 mL, such as 0.3 mL) is performed on each human eye separately, using standard vitreoretinal techniques for subretinal surgery. In certain embodiments, the subject is given a short-term corticosteroid regimen of oral prednisone (or the equivalent), before and/or after the subretinal injection to each eye in need to treatment.

The therapeutically effective dose for the subject viral particles in a mammal other than mouse (e.g., in human), can be based on the effective dose tested in mouse, which is about 1E+7 (1×10⁷ vg/mouse eye) to about 3E+9 (3×10⁹ vg/mouse eye), such as, about 1E+7 (1×10⁷ vg/mouse eye), about 3E+7 (3×10⁷ vg/mouse eye), about 1E+8 (1×10⁸ vg/mouse eye), about 3E+8 (3×10⁸ vg/mouse eye), about 1E+9 (1×10⁹ vg/mouse eye), about 3E+9 (3×10⁹ vg/mouse eye). Factors such as the size of the human eye, the area of the retinal pigment epithelial cell layer in the human eye, and the number of RPE cells in a human eye can be used to determine the dosage level and interval, plus physician discretion to increase or decrease does based on the specific situation of a patient.

In some embodiments, the vectors, e.g., plasmids or viral vectors, are delivered to the tissue of interest by, e.g., intramuscular injection, intravenous administration, transdermal administration, intranasal administration, oral administration, or mucosal administration. Such delivery may be either via a single dose, or multiple doses. One skilled in the art understands that the actual dosage to be delivered herein may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

In certain embodiments, the delivery is via adenoviruses, which can be at a single dose containing at least 1×105 particles (also referred to as particle units, pu) of adenoviruses. In some embodiments, the dose preferably is at least about 1×10⁶ particles, at least about 1×10⁷ particles, at least about 1×10⁸ particles, and at least about 1×10⁹ particles of the adenoviruses. The delivery methods and the doses are described, e.g., in WO 2016205764 A1 and U.S. Pat. No. 8,454,972 B2, both of which are incorporated herein by reference in the entirety.

In some embodiments, the delivery is via plasmids. The dosage can be a sufficient number of plasmids to elicit a response. In some cases, suitable quantities of plasmid DNA in plasmid compositions can be from about 0.1 to about 2 mg. Plasmids will generally include (i) a promoter; (ii) a sequence encoding a gene of interest, e.g., an RPE65 polypeptide, or variant thereof, operably linked to the promoter; (iii) a selectable marker; (iv) an origin of replication; and (v) a transcription terminator downstream of and operably linked to (ii). The frequency of administration is within the ambit of the medical or veterinary practitioner (e.g., physician, veterinarian), or a person skilled in the art.

In another embodiment, the delivery is via liposomes or lipofection formulations and the like, and can be prepared by methods known to those skilled in the art. Such methods are described, for example, in WO 2016205764 and U.S. Pat. Nos. 5,593,972; 5,589,466; and 5,580,859; each of which is incorporated herein by reference in its entirety.

In some embodiments, the delivery is via nanoparticles or exosomes. For example, exosomes have been shown to be particularly useful in delivery RNA.

8. Kits Another aspect of the disclosure provides a kit, comprising an rAAV vector genome or an rAAV viral particle described herein comprising a polynucleotide encoding a gene of interest, e.g., an RPE65 polypeptide or functional variants thereof, vectors encompassing the same, or host encompassing the same.

In certain embodiments, the kit further comprises an instruction to use the components encompassed therein, and/or instructions for combining with additional components that may be available elsewhere.

In certain embodiments, the kit further comprises one or more nucleotides, such as nucleotide(s) corresponding to those useful to insert the guide RNA coding sequence into a vector and operably linking the coding sequence to one or more control elements of the vector.

In certain embodiments, the kit further comprises one or more buffers that may be used to dissolve any of the components, and/or to provide suitable reaction conditions for one or more of the components.

Such buffers may include one or more of PBS, HEPES, Tris, MOPS, Na₂CO₃, NaHCO₃, NaB, or combinations thereof. In certain embodiments, the reaction condition includes a proper pH, such as a basic pH. In certain embodiments, the pH is between 7-10.

In certain embodiments, any one or more of the kit components may be stored in a suitable container.

### 9. Pharmaceutical Compositions

Another aspect of the disclosure provides pharmaceutical compositions comprising an isolated nucleic acid or rAAV as described herein and a pharmaceutically acceptable carrier.

As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol.

Specifically contemplated routes include subretinal injection, such as subretinal injection following a vitrectomy.

For example, in certain embodiments, the rAAV viral particle encoding RPE65 is delivered to an affected eye of a patient via subretinal injection, e.g., subretinal injection of the rAAV viral particles of the disclosure after vitrectomy. The vitrectomy may be a standard three-port pars plana vitrectomy, with removal of the posterior cortical vitreous (see Maguire et al., Safety and efficacy of gene transfer for Leber congenital amaurosis. N Engl J Med. 358:2240-2248, 2008, incorporated by reference).

In certain embodiments, each injection comprises from 1.5×10¹⁰ vector genomes (e.g., at a concentration of about 1.0×10⁸ vg/μL) to 1.5×10¹¹ vector genomes (e.g., at a concentration of about 5.0×10⁸ vg/μL).

In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In certain embodiments, the compound or pharmaceutical composition described herein is suitable for topical administration to the eye of a subject.

### EXAMPLES

### Example 1 rAAV Viral Particle Restored Visual Function

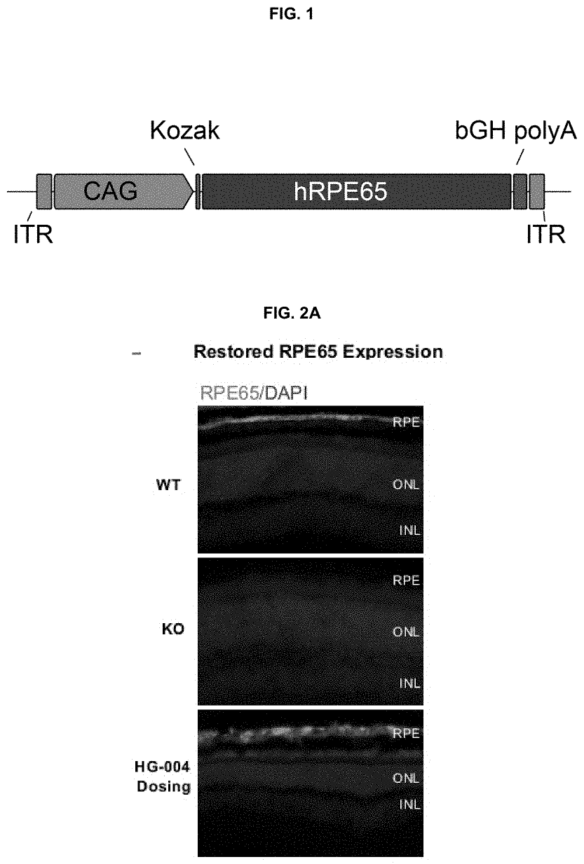

A representative AAV9 vector (AAV9-CAG-hRPE65, “HG004”) with an AAV9 serotype capsid encapsulating a vector genome encoding human RPE65 gene was constructed as a new therapy to treat LCA2 (FIG. 1). The vector genome (of SEQ ID NO: 1 from 5′ ITR to 3′ ITR) included a human RPE65 coding sequence (SEQ ID NO: 2) encoding human RPE65 polypeptide (SEQ ID NO: 3), a CAG promoter (SEQ ID NO: 4) operably linked to and drived the transcription of the human RPE65 coding sequence, a Kozak sequence (SEQ ID NO: 6) upstream of and immediately 5′ to the human RPE65 coding sequence, a bGH polyA sequence (SEQ ID NO: 5) downstream of the human RPE65 coding sequence, flanked by a 5′ ITR (SEQ ID NO: 7) and a 3′ ITR (SEQ ID NO: 8), which were derived from AAV2.

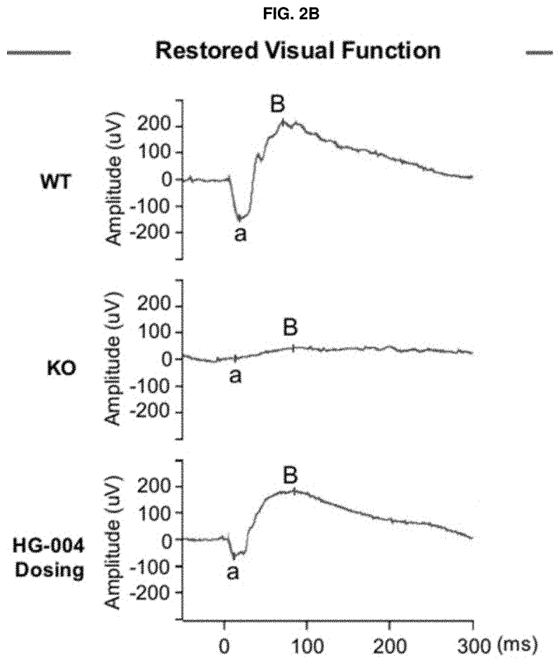

Firstly, to assess the effect of AAV9-CAG-hRPE65 (“HG004”) on LCA2 therapy, RPE65 knockout mice (RPE65′) were generated to serve as LCA2 mouse model by RPE65 exon 3 deletion. RPE65 expression was successfully knocked out in the retinal pigment epithelium cells (FIG. 2A), based on visual function measured by ERG (visual electrophysiology) compared with the corresponding wild type (RPE65^{+/+}) mouse (FIG. 2B and FIG. 2C). RPE65^{-/-} mice showed at least a 5-fold decrease in B-wave signal compared with the corresponding wild type (RPE65^{+/+}) mouse (FIG. 2C).

It was observed that the administration of AAV9-CAG-hRPE65 (“HG004”) to RPE65^{-/-} mice restored the expression of RPE65 in the retinal pigment epithelium cells (FIG. 2A) and restored visual function measured by ERG to a comparable level to the corresponding wild type (RPE65^{+/+}) mouse (FIG. 2B).

AAV9-CAG-hRPE65 (“HG-004”) administered at different doses (3×10⁶ or 3E+6, 1×10⁷ or 1E+7, 3×10⁷ or 3E+7, 1×10⁸ or 1E+8, 3×10⁸ or 3E+8, 1×10⁹ or 1E+9, 3×10⁹ or 3E+9, and 1×10¹⁰ or 1E+10 vg/eye (“vg”, viral genomes), or “Doses 1-8,” respectively) was subretinally injected into one eye of RPE65^{-/-} mice, and ERG were measured 3, 6, 9, and 14 weeks post dosing. The contralateral eye was measured as a control with PBS subretinal injection.

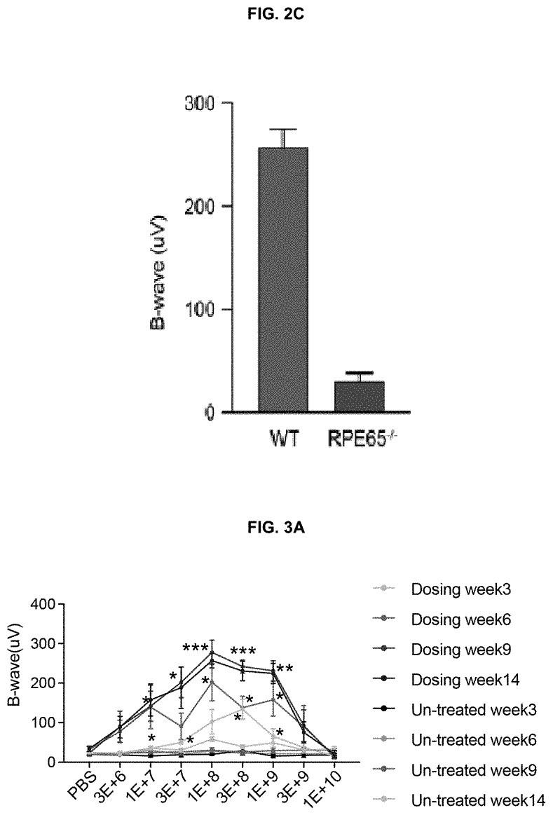

The results showed the effective dose range of from 1E+7 vg/eye (Dose 2) to 3E+9 vg/eye (Dose 7). The restoration of visual function maximized at 9 weeks post dosing (B-wave, 277.84±25.55 uv, at a dose of 1E+8 vg/eye (Dose 4), about ˜70% of the B-wave value of the corresponding wild type (RPE65^{+/+}) mouse (data not shown)), and the therapeutical effect lasted till at least 14 weeks and more post dosing (FIG. 3A and FIG. 3B). For example, the administration of AAV9-CAG-hRPE65 (“HG-004”) at a dose of 3E+7 (Dose 3) restored vision about 50%, 60%, 70%, and 72% at weeks 3, 6, 9, 14, respectively.

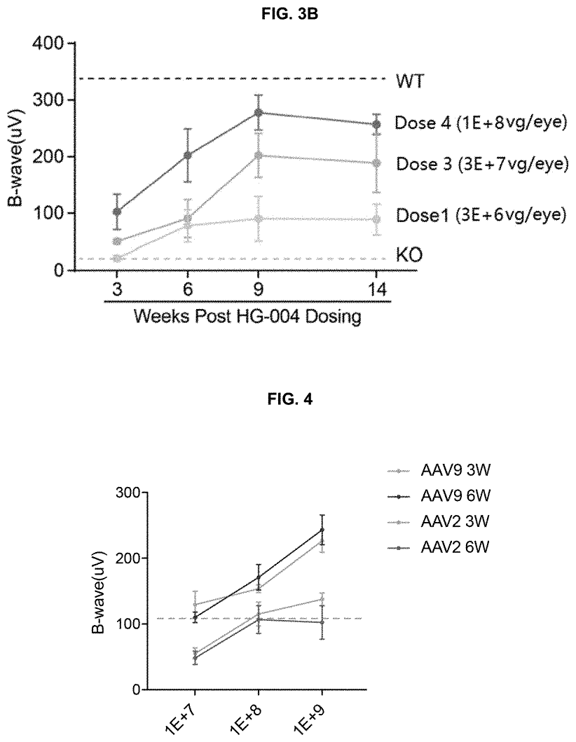

To further investigate the optimization effect of AAV9-CAG-hRPE65 (“HG-004”), the same vector genome encoding hRPE65 gene was delivered using AAV2 vector (with an AAV2 capsid) instead of AAV9 for direct comparison. Surprisingly, AAV9-CAG-hRPE65 achieved similar visual function restoration effect with about 1/10 to about 1/100 of the dose of AAV2-CAG-hRPE65 (FIG. 4), indicating a surprising and unexpected result that AAV9 would deliver an RPE65 transgene more effectively than AAV2.

The data showed that AAV9-CAG-hRPE65 (“HG-004”) successfully rescued visual function in an RPE65 knockout LCA2 mouse model, and more surprisingly, the AAV9 vector was more effective than the reported strategy using AAV2 vector. Subretinally injected AAV9-CAG-hRPE65 (“HG-004”) into the RPE65 knockout LCA2 mouse model restored ˜70% of wild type (RPE65^{+/+}) visual function. The results indicate a new and promising clinical therapy for LCA2.

Methods

Animal

RPE65^{-/-} were generated using CRISPR-Cas9 and bred onto C57BL/6J background for at least one generation. Animals were housed in in-house animal facility on 12h:12h light/dark cycle. Food and water were given ad libitum. All experimental protocols were approved by the Animal Care and Use Committee.

AAV Vector Preparation

Recombinant AAV9 and AAV2 viral particles were generated by triple transfection of HEK293T cells using polyethylenimine (PEI). Viral particles were harvested from the media at 72 hours post transfection and from the cells and media at 120 hours. Cell pellets were resuspended in 10 mM Tris with 10 mM MgCl₂ and 150 mM sodium chloride, pH 7.6, freeze-thawed three times, and treated with 125 U/mL Benzonase (Sigma) at 37° C. for at least 1 hr. Viral media was concentrated by precipitation with 10% polyethylene glycol 8000 (Sigma-Aldrich) with 625 mM sodium chloride, resuspended in PBS with 0.001% Pluronic™ F-68 Non-ionic Surfactant, and then added to the lysates. The combined stocks were then adjusted to 100 mM NaCl, incubated at 37° C. for 1 hr, and clarified by centrifugation at 2,000 g. The clarified stocks were then purified over iodixanol (Optiprep, Sigma; D1556) step gradients (15%, 25%, 40% and 58%). Viral particles were concentrated and formulated in PBS with 0.001% Pluronic™ F-68 Non-ionic Surfactant. Virus titers were determined by measuring the number of DNaseI resistant vector genomes using qPCR with linearized genome plasmid as a standard.

Subretinal Injection

4-8 weeks old mice were anesthetized with mixture of zoletil (60 μg/g) and xylazine (10 μg/g). A small hole at slight posterior to the limbus were punctured with a sterile 31 G ½ needle following pupil dilation. About 1 μL rAAV injection were subretinal injected through the hole using a Hamilton syringe with a 33G blunt needle.

Fluorescent Immunostaining

RPE65^{-/-} (LCA2 model) or RPE65^{+/+}(WT) mice were anesthetized and perfused with PBS followed by ice-cold 4% paraformaldehyde. Eyes were isolated and post-fixed with 4% paraformaldehyde overnight. For retina complex, a knife cut was made on the cornea before eye dehydration and the lens was removed before embedded. Tissues were sectioned at 20 μm thickness using a freezing microtome (Leica CM1950) and sections were mounted to slide directly. Slides were baked at 60° C. for 1-2 hours, followed by incubating with RPE65 antibody (1:000, MAB5428, Millipore) at 4° C. overnight. The second day slides were washed with PBS and then incubated with donkey anti-mouse antibody (1:1000, 715-545-151, Jackson ImmunoReseach labs) and DAPI (1:1000, D3571, Invitrogen) for 2 hours. Afterward, images were captured with Nikon Ni-E microscope.

ERG

Mice were dark adapted for more than 2 hours and then anesthetized with mixture of zoletil (60 μg/g) and xylazine (10 μg/g). One drop of Tropicamide Phenylephrine was placed in both eyes to induce mydriasis. Mice were placed on a heating pad (37° C.) and the electrodes were attached to the corneas. Scotopic 3.0 ERG were then tested under 3.0 cd·s/m² stimuli.

### Exemplary Sequences

CAG-hRPE65 coding sequence (ITR-to-ITR)
(SEQ ID NO: 1)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCTGCGGCCATC
GATGGCGCGCCGGATTCGACATTGATTATTGACTAGTTATTAATAGTAAT
CAATTACGGGGTCATTAGTTCATAGCCCATATATGGAGTTCCGCGTTACA
TAACTTACGGTAAATGGCCCGCCTGGCTGACCGCCCAACGACCCCCGCCC
ATTGACGTCAATAATGACGTATGTTCCCATAGTAACGCCAATAGGGACTT
TCCATTGACGTCAATGGGTGGAGTATTTACGGTAAACTGCCCACTTGGCA
GTACATCAAGTGTATCATATGCCAAGTACGCCCCCTATTGACGTCAATGA
CGGTAAATGGCCCGCCTGGCATTATGCCCAGTACATGACCTTATGGGACT
TTCCTACTTGGCAGTACATCTACGTATTAGTCATCGCTATTACCATGGTC
GAGGTGAGCCCCACGTTCTGCTTCACTCTCCCCATCTCCCCCCCCTCCCC
ACCCCCAATTTTGTATTTATTTATTTTTTAATTATTTTGTGCAGCGATGG
GGGCGGGGGGGGGGGGGGGGCGCGCGCCAGGCGGGGCGGGGCGGGGCGAG
GGGCGGGGCGGGGCGAGGCGGAGAGGTGCGGCGGCAGCCAATCAGAGCGG
CGCGCTCCGAAAGTTTCCTTTTATGGCGAGGCGGCGGCGGCGGCGGCCCT
ATAAAAAGCGAAGCGCGCGGCGGGCGGGAGTCGCTGCGTTGCCTTCGCCC
CGTGCCCCGCTCCGCGCCGCCTCGCGCCGCCCGCCCCGGCTCTGACTGAC
CGCGTTACTCCCACAGGTGAGCGGGCGGGACGGCCCTTCTCCTCCGGGCT
GTAATTAGCGCTTGGTTTAATGACGGCTCGTTTCTTTTCTGTGGCTGCGT
GAAAGCCTTAAAGGGCTCCGGGAGGGCCCTTTGTGCGGGGGGGAGCGGCT
CGGGGGGTGCGTGCGTGTGTGTGTGCGTGGGGAGCGCCGCGTGCGGCCCG
CGCTGCCCGGCGGCTGTGAGCGCTGCGGGCGCGGCGCGGGGCTTTGTGCG
CTCCGCGTGTGCGCGAGGGGAGCGCGGCCGGGGGCGGTGCCCCGCGGTGC
GGGGGGGCTGCGAGGGGAACAAAGGCTGCGTGCGGGGTGTGTGCGTGGGG
GGGTGAGCAGGGGGTGTGGGCGCGGCGGTCGGGCTGTAACCCCCCCCTGC
ACCCCCCTCCCCGAGTTGCTGAGCACGGCCCGGCTTCGGGTGCGGGGCTC
CGTGCGGGGCGTGGCGCGGGGCTCGCCGTGCCGGGCGGGGGGTGGCGGCA
GGTGGGGGTGCCGGGCGGGGCGGGGCCGCCTCGGGCCGGGGAGGGCTCGG
GGGAGGGGCGCGGCGGCCCCGGAGCGCCGGCGGCTGTCGAGGCGCGGCGA
GCCGCAGCCATTGCCTTTTATGGTAATCGTGCGAGAGGGCGCAGGGACTT
CCTTTGTCCCAAATCTGGCGGAGCCGAAATCTGGGAGGCGCCGCCGCACC
CCCTCTAGCGGGCGCGGGCGAAGCGGTGCGGCGCCGGCAGGAAGGAAATG
GGCGGGGAGGGCCTTCGTGCGTCGCCGCGCCGCCGTCCCCTTCTCCATCT
CCAGCCTCGGGGCTGCCGCAGGGGGACGGCTGCCTTCGGGGGGGACGGGG
CAGGGCGGGGTTCGGCTTCTGGCGTGTGACCGGCGGCTCTAGAGCCTCTG
CTAACCATGTTCATGCCTTCTTCTTTTTCCTACAGATCCTTAATGCCACC
ATGTCTATCCAGGTTGAGCATCCTGCTGGTGGTTACAAGAAACTGTTTGA
AACTGTGGAGGAACTGTCCTCGCCGCTCACAGCTCATGTAACAGGCAGGA
TCCCCCTCTGGCTCACCGGCAGTCTCCTTCGATGTGGGCCAGGACTCTTT
GAAGTTGGATCTGAGCCATTTTACCACCTGTTTGATGGGCAAGCCCTCCT
GCACAAGTTTGACTTTAAAGAAGGACATGTCACATACCACAGAAGGTTCA
TCCGCACTGATGCTTACGTACGGGCAATGACTGAGAAAAGGATCGTCATA
ACAGAATTTGGCACCTGTGCTTTCCCAGATCCCTGCAAGAATATATTTTC
CAGGTTTTTTTCTTACTTTCGAGGAGTAGAGGTTACTGACAATGCCCTTG
TTAATGTCTACCCAGTGGGGGAAGATTACTACGCTTGCACAGAGACCAAC
TTTATTACAAAGATTAATCCAGAGACCTTGGAGACAATTAAGCAGGTTGA
TCTTTGCAACTATGTCTCTGTCAATGGGGCCACTGCTCACCCCCACATTG
AAAATGATGGAACCGTTTACAATATTGGTAATTGCTTTGGAAAAAATTTT
TCAATTGCCTACAACATTGTAAAGATCCCACCACTGCAAGCAGACAAGGA
AGATCCAATAAGCAAGTCAGAGATCGTTGTACAATTCCCCTGCAGTGACC
GATTCAAGCCATCTTACGTTCATAGTTTTGGTCTGACTCCCAACTATATC
GTTTTTGTGGAGACACCAGTCAAAATTAACCTGTTCAAGTTCCTTTCTTC
ATGGAGTCTTTGGGGAGCCAACTACATGGATTGTTTTGAGTCCAATGAAA
CCATGGGGGTTTGGCTTCATATTGCTGACAAAAAAAGGAAAAAGTACCTC
AATAATAAATACAGAACTTCTCCTTTCAACCTCTTCCATCACATCAACAC
CTATGAAGACAATGGGTTTCTGATTGTGGATCTCTGCTGCTGGAAAGGAT
TTGAGTTTGTTTATAATTACTTATATTTAGCCAATTTACGTGAGAACTGG
GAAGAGGTGAAAAAAAATGCCAGAAAGGCTCCCCAACCTGAAGTTAGGAG
ATATGTACTTCCTTTGAATATTGACAAGGCTGACACAGGCAAGAATTTAG
TCACGCTCCCCAATACAACTGCCACTGCAATTCTGTGCAGTGACGAGACT
ATCTGGCTGGAGCCTGAAGTTCTCTTTTCAGGGCCTCGTCAAGCATTTGA
GTTTCCTCAAATCAATTACCAGAAGTATTGTGGGAAACCTTACACATATG
CGTATGGACTTGGCTTGAATCACTTTGTTCCAGATAGGCTCTGTAAGCTG
AATGTCAAAACTAAAGAAACTTGGGTTTGGCAAGAGCCTGATTCATACCC
ATCAGAACCCATCTTTGTTTCTCACCCAGATGCCTTGGAAGAAGATGATG
GTGTAGTTCTGAGTGTGGTGGTGAGCCCAGGAGCAGGACAAAAGCCTGCT
TATCTCCTGATTCTGAATGCCAAGGACTTAAGTGAAGTTGCCCGGGCTGA
AGTGGAGATTAACATCCCTGTCACCTTTCATGGACTGTTCAAAAAATCTT
GACTAACTGTGCCTTCTAGTTGCCAGCCATCTGTTGTTTGCCCCTCCCCC
GTGCCTTCCTTGACCCTGGAAGGTGCCACTCCCACTGTCCTTTCCTAATA
AAATGAGGAAATTGCATCGCATTGTCTGAGTAGGTGTCATTCTATTCTGG
GGGGTGGGGTGGGGCAGGACAGCAAGGGGGAGGATTGGGAAGACAATAGC
AGGCATGCTGGGGATGCGGTGGGCTCTATGGCTTGCGGCCGCGTCGGCCG
CAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCC
GGGCGGCCTCAGTGAGCGAGCGAGCGCGCAGCTGCCTGCAGG
hRPE65 coding sequence
(SEQ ID NO: 2)
ATGTCTATCCAGGTTGAGCATCCTGCTGGTGGTTACAAGAAACTGTTTGA
AACTGTGGAGGAACTGTCCTCGCCGCTCACAGCTCATGTAACAGGCAGGA
TCCCCCTCTGGCTCACCGGCAGTCTCCTTCGATGTGGGCCAGGACTCTTT
GAAGTTGGATCTGAGCCATTTTACCACCTGTTTGATGGGCAAGCCCTCCT
GCACAAGTTTGACTTTAAAGAAGGACATGTCACATACCACAGAAGGTTCA
TCCGCACTGATGCTTACGTACGGGCAATGACTGAGAAAAGGATCGTCATA
ACAGAATTTGGCACCTGTGCTTTCCCAGATCCCTGCAAGAATATATTTTC
CAGGTTTTTTTCTTACTTTCGAGGAGTAGAGGTTACTGACAATGCCCTTG
TTAATGTCTACCCAGTGGGGGAAGATTACTACGCTTGCACAGAGACCAAC
TTTATTACAAAGATTAATCCAGAGACCTTGGAGACAATTAAGCAGGTTGA
TCTTTGCAACTATGTCTCTGTCAATGGGGCCACTGCTCACCCCCACATTG
AAAATGATGGAACCGTTTACAATATTGGTAATTGCTTTGGAAAAAATTTT
TCAATTGCCTACAACATTGTAAAGATCCCACCACTGCAAGCAGACAAGGA
AGATCCAATAAGCAAGTCAGAGATCGTTGTACAATTCCCCTGCAGTGACC
GATTCAAGCCATCTTACGTTCATAGTTTTGGTCTGACTCCCAACTATATC
GTTTTTGTGGAGACACCAGTCAAAATTAACCTGTTCAAGTTCCTTTCTTC
ATGGAGTCTTTGGGGAGCCAACTACATGGATTGTTTTGAGTCCAATGAAA
CCATGGGGGTTTGGCTTCATATTGCTGACAAAAAAAGGAAAAAGTACCTC
AATAATAAATACAGAACTTCTCCTTTCAACCTCTTCCATCACATCAACAC
CTATGAAGACAATGGGTTTCTGATTGTGGATCTCTGCTGCTGGAAAGGAT
TTGAGTTTGTTTATAATTACTTATATTTAGCCAATTTACGTGAGAACTGG
GAAGAGGTGAAAAAAAATGCCAGAAAGGCTCCCCAACCTGAAGTTAGGAG
ATATGTACTTCCTTTGAATATTGACAAGGCTGACACAGGCAAGAATTTAG
TCACGCTCCCCAATACAACTGCCACTGCAATTCTGTGCAGTGACGAGACT
ATCTGGCTGGAGCCTGAAGTTCTCTTTTCAGGGCCTCGTCAAGCATTTGA
GTTTCCTCAAATCAATTACCAGAAGTATTGTGGGAAACCTTACACATATG
CGTATGGACTTGGCTTGAATCACTTTGTTCCAGATAGGCTCTGTAAGCTG
AATGTCAAAACTAAAGAAACTTGGGTTTGGCAAGAGCCTGATTCATACCC
ATCAGAACCCATCTTTGTTTCTCACCCAGATGCCTTGGAAGAAGATGATG
GTGTAGTTCTGAGTGTGGTGGTGAGCCCAGGAGCAGGACAAAAGCCTGCT
TATCTCCTGATTCTGAATGCCAAGGACTTAAGTGAAGTTGCCCGGGCTGA
AGTGGAGATTAACATCCCTGTCACCTTTCATGGACTGTTCAAAAAATCTT
GA
hRPE65 amino acid sequence
(SEQ ID NO: 3)
MSIQVEHPAGGYKKLFETVEELSSPLTAHVTGRIPLWLTGSLLRCGPGLF
EVGSEPFYHLFDGQALLHKFDFKEGHVTYHRRFIRTDAYVRAMTEKRIVI
TEFGTCAFPDPCKNIFSRFFSYFRGVEVTDNALVNVYPVGEDYYACTETN
FITKINPETLETIKQVDLCNYVSVNGATAHPHIENDGTVYNIGNCFGKNF
SIAYNIVKIPPLQADKEDPISKSEIVVQFPCSDRFKPSYVHSFGLTPNYI
VFVETPVKINLFKFLSSWSLWGANYMDCFESNETMGVWLHIADKKRKKYL
NNKYRTSPFNLFHHINTYEDNGFLIVDLCCWKGFEFVYNYLYLANLRENW
EEVKKNARKAPQPEVRRYVLPLNIDKADTGKNLVTLPNTTATAILCSDET
IWLEPEVLFSGPRQAFEFPQINYQKYCGKPYTYAYGLGLNHFVPDRLCKL
NVKTKETWVWQEPDSYPSEPIFVSHPDALEEDDGVVLSVVVSPGAGQKPA
YLLILNAKDLSEVARAEVEINIPVTFHGLFKKS
CAG promoter
(SEQ ID NO: 4)
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGAGTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATGGTCGAGGTGAGCCCCACGTT
CTGCTTCACTCTCCCCATCTCCCCCCCCTCCCCACCCCCAATTTTGTATT
TATTTATTTTTTAATTATTTTGTGCAGCGATGGGGGCGGGGGGGGGGGGG
GGGCGCGCGCCAGGCGGGGCGGGGCGGGGCGAGGGGCGGGGCGGGGCGAG
GCGGAGAGGTGCGGCGGCAGCCAATCAGAGCGGCGCGCTCCGAAAGTTTC
CTTTTATGGCGAGGCGGCGGCGGCGGCGGCCCTATAAAAAGCGAAGCGCG
CGGCGGGCGGGAGTCGCTGCGTTGCCTTCGCCCCGTGCCCCGCTCCGCGC
CGCCTCGCGCCGCCCGCCCCGGCTCTGACTGACCGCGTTACTCCCACAGG
TGAGCGGGCGGGACGGCCCTTCTCCTCCGGGCTGTAATTAGCGCTTGGTT
TAATGACGGCTCGTTTCTTTTCTGTGGCTGCGTGAAAGCCTTAAAGGGCT
CCGGGAGGGCCCTTTGTGCGGGGGGGAGCGGCTCGGGGGGTGCGTGCGTG
TGTGTGTGCGTGGGGAGCGCCGCGTGCGGCCCGCGCTGCCCGGCGGCTGT
GAGCGCTGCGGGCGCGGCGCGGGGCTTTGTGCGCTCCGCGTGTGCGCGAG
GGGAGCGCGGCCGGGGGCGGTGCCCCGCGGTGCGGGGGGGCTGCGAGGGG
AACAAAGGCTGCGTGCGGGGTGTGTGCGTGGGGGGGTGAGCAGGGGGTGT
GGGCGCGGCGGTCGGGCTGTAACCCCCCCCTGCACCCCCCTCCCCGAGTT
GCTGAGCACGGCCCGGCTTCGGGTGCGGGGCTCCGTGCGGGGCGTGGCGC
GGGGCTCGCCGTGCCGGGCGGGGGGTGGCGGCAGGTGGGGGTGCCGGGCG
GGGCGGGGCCGCCTCGGGCCGGGGAGGGCTCGGGGGAGGGGCGCGGCGGC
CCCGGAGCGCCGGCGGCTGTCGAGGCGCGGCGAGCCGCAGCCATTGCCTT
TTATGGTAATCGTGCGAGAGGGCGCAGGGACTTCCTTTGTCCCAAATCTG
GCGGAGCCGAAATCTGGGAGGCGCCGCCGCACCCCCTCTAGCGGGCGCGG
GCGAAGCGGTGCGGCGCCGGCAGGAAGGAAATGGGCGGGGAGGGCCTTCG
TGCGTCGCCGCGCCGCCGTCCCCTTCTCCATCTCCAGCCTCGGGGCTGCC
GCAGGGGGACGGCTGCCTTCGGGGGGGACGGGGCAGGGCGGGGTTCGGCT
TCTGGCGTGTGACCGGCGGCTCTAGAGCCTCTGCTAACCATGTTCATGCC
TTCTTCTTTTTCCTACAGATCC
bGH polyA signal sequence
(SEQ ID NO: 5)
CTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGA
GGAAATTGCATCGCATTGTCTGAGTAGGTGTCATTCTATTCTGGGGGGTG
GGGTGGGGCAGGACAGCAAGGGGGAGGATTGGGAAGACAATAGCAGGCAT
GCTGGGGATGCGGTGGGCTCTATGG
Kozak sequence
(SEQ ID NO: 6)
GCCACC
5′ ITR Sequence based on AAV2 5′ ITR
(SEQ ID NO: 7)
CCTGCAGGCAGCTGCGCGCTCGCTCGCTCACTGAGGCCGCCCGGGCAAAG
CCCGGGCGTCGGGCGACCTTTGGTCGCCCGGCCTCAGTGAGCGAGCGAGC
GCGCAGAGAGGGAGTGGCCAACTCCATCACTAGGGGTTCCT
3′ ITR Sequence based on AAV2 3′ ITR
(SEQ ID NO: 8)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG
AAV9 VP1 Capsid Sequence
(SEQ ID NO: 9)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL
(SEQ ID NO: 11)
CCTGCAGGCAG
(SEQ ID NO: 12)
TTAATTAAGG
(SEQ ID NO: 13)
AAAGCCCGGGC

1. A recombinant adeno-associated virus (rAAV) viral particle comprising an AAV9 serotype capsid and a vector genome encoding an RPE65 (e.g., hRPE65) polypeptide, such as a polynucleotide at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO: 1.

2. The rAAV viral particle of claim 1, wherein the vector genome comprises:
a) a 5′ inverted terminal repeat (ITR);
b) an RPE65 polynucleotide encoding an RPE65 polypeptide, wherein the RPE65 polynucleotide comprises a polynucleotide sequence of SEQ ID NO: 2 or having a sequence identity of at least 90%, 95%, 96%, 97%, 98%, 99%, 99.2%, 99.4%, 99.6%, or 99.8% to the polynucleotide sequence of SEQ ID NO: 2;
c) a promoter operably linked to and drives the transcription of the RPE65 polynucleotide;
d) an optional Kozak sequence upstream of the RPE65 polynucleotide and downstream of the promoter;
e) a polyA signal sequence; and
f) a 3′ ITR,
optionally, wherein the RPE65 polypeptide has the amino acid sequence of SEQ ID NO: 3.

3. The rAAV viral particle of claim 2, wherein the 5′ ITR and 3′ ITR are derived from AAV2 or AAV9, optionally, the 5′ ITR comprises the nucleotide sequence of SEQ ID NO: 7, and/or and the 3′ ITR comprises the nucleotide sequence of SEQ ID NO: 8.

4. The rAAV viral particle of claim 2, wherein the promoter is a ubiquitous promoter, a tissue-specific promoter, a constitutive promoter, or an inducible promoter.

5-7. (canceled)

8. The rAAV viral particle of claim 2, wherein the promoter is selected from the group consisting of a pol I promoter, a pol II promoter, a pol III promoter, a T7 promoter, a U6 promoter, a H1 promoter, retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a β glucuronidase (GUSB) promoter, a cytomegalovirus (CMV) immediate-early (IE) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, or a functional fragment thereof.

9. The rAAV viral particle of claim 8, wherein the promoter is the CAG promoter.

10. The rAAV viral particle of claim 9, wherein the CAG promoter comprises a sequence having a sequence identity of at least 95%, 96%, 97%, 98%, or 99% to SEQ ID NO: 4.

11. The rAAV viral particle of claim 10, wherein the CAG promoter comprises, consists essentially of, or consists of SEQ ID NO: 4.

12. The rAAV viral particle of claim 2, wherein the polyA signal sequence is selected from the group consisting of bovine growth hormone polyadenylation signal sequence (bGH polyA), a small polyA signal sequence (SPA), a human growth hormone polyadenylation signal sequence (hGH polyA), a rabbit beta globin polyA signal sequence (rBG polyA), an SV40 polyA signal sequence (SV40 polyA), or a variant thereof.

13. The rAAV viral particle of claim 12, wherein the polyA signal sequence is the bGH polyA.

14. The rAAV viral particle of claim 13, wherein the bGH polyA comprises, consists essentially of, or consists of SEQ ID NO: 5.

15. The rAAV viral particle of claim 2, wherein the Kozak sequence is GCCACC (SEQ ID NO: 6) or a sequence comprising at most 1, 2, 3, or 4 nucleotide differences from GCCACC (SEQ ID NO: 6), and optionally wherein the last three nucleotide is ACC or GCC.

16. The rAAV viral particle of claim 1, wherein the vector genome comprises, in 5′ to 3′ direction,
(1) a 5′ ITR of SEQ ID NO: 7,
(2) a CAG promoter of SEQ ID NO: 4,
(3) a Kozak sequence of GCCACC (SEQ ID NO: 6),
(4) a hRPE65 polynucleotide sequence of SEQ ID NO: 2,
(5) a bGH polyA signal sequence of SEQ ID NO: 5, and
(6) a 3′ ITR of SEQ ID NO: 8,
with an optional linker between (1) and (2), between (2) and (3), between (3) and (4), between (4) and (5), and/or between (5) and (6);
optionally wherein the vector genome comprises, consists essentially of, or consists of SEQ ID NO: 1, or a polynucleotide having a sequence identity of at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% to SEQ ID NO: 1, encoding an RPE65 polypeptide having a sequence identity of at least 95%, 96%, 97%, 98%, or 99% to SEQ ID NO: 3 (e.g., 100% identical to SEQ ID NO: 3).

17. The rAAV viral particle of claim 16, wherein the vector genome has a sequence identity of at least 99% to SEQ ID NO: 1.

18. The rAAV viral particle of claim 16, wherein the vector genome consists of SEQ ID NO: 1.

19. The rAAV viral particle of claim 1, wherein the AAV9 serotype capsid comprises AAV9 VP1, AAV9 VP2, and AAV9 VP3; or VP1, VP2, and VP3 variants independently having a sequence identity of at least 90%, 91%, 92%, 93%, 94%, 95%, 96%, 97%, 98%, or 99% thereto, respectively.

20. The rAAV viral particle of claim 19, wherein the AAV9 serotype capsid comprises AAV9 VP1 (SEQ ID NO: 9), AAV9 VP2, and AAV9 VP3.

21. A pharmaceutical composition comprising the rAAV viral particle of claim 1, and a pharmaceutically acceptable excipient.

22. A method of treating a RPE65-associated eye disease or disorder (e.g., (human) RPE65-deficient) in a subject in need thereof, comprising administering to the subject a therapeutically effective amount of the rAAV viral particle of claim 1, wherein the rAAV viral particle specifically induces expression of the RPE65 polypeptide from the vector genome of the rAAV viral particle (e.g., in retinal pigment epithelial (RPE) cells).

23-32. (canceled)


SEQUENCE LISTING (13 sequences)

SEQ ID NO: 1 - DNA, 3842 bp, synthetic construct
  misc_feature 1..3842: note = source = /note="Description of Artificial Sequence: Syntheticpolynucleotide"
  source 1..3842: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccatcgatggcgcgccggattcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatccttaatgccaccatgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaatgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttgactaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttgcggccgcgtcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 1602 bp, Homo sapiens
  source 1..1602: mol_type = genomic DNA
atgtctatccaggttgagcatcctgctggtggttacaagaaactgtttgaaactgtggaggaactgtcctcgccgctcacagctcatgtaacaggcaggatccccctctggctcaccggcagtctccttcgatgtgggccaggactctttgaagttggatctgagccattttaccacctgtttgatgggcaagccctcctgcacaagtttgactttaaagaaggacatgtcacataccacagaaggttcatccgcactgatgcttacgtacgggcaatgactgagaaaaggatcgtcataacagaatttggcacctgtgctttcccagatccctgcaagaatatattttccaggtttttttcttactttcgaggagtagaggttactgacaatgcccttgttaatgtctacccagtgggggaagattactacgcttgcacagagaccaactttattacaaagattaatccagagaccttggagacaattaagcaggttgatctttgcaactatgtctctgtcaatggggccactgctcacccccacattgaaaatgatggaaccgtttacaatattggtaattgctttggaaaaaatttttcaattgcctacaacattgtaaagatcccaccactgcaagcagacaaggaagatccaataagcaagtcagagatcgttgtacaattcccctgcagtgaccgattcaagccatcttacgttcatagttttggtctgactcccaactatatcgtttttgtggagacaccagtcaaaattaacctgttcaagttcctttcttcatggagtctttggggagccaactacatggattgttttgagtccaatgaaaccatgggggtttggcttcatattgctgacaaaaaaaggaaaaagtacctcaataataaatacagaacttctcctttcaacctcttccatcacatcaacacctatgaagacaatgggtttctgattgtggatctctgctgctggaaaggatttgagtttgtttataattacttatatttagccaatttacgtgagaactgggaagaggtgaaaaaaaatgccagaaaggctccccaacctgaagttaggagatatgtacttcctttgaatattgacaaggctgacacaggcaagaatttagtcacgctccccaatacaactgccactgcaattctgtgcagtgacgagactatctggctggagcctgaagttctcttttcagggcctcgtcaagcatttgagtttcctcaaatcaattaccagaagtattgtgggaaaccttacacatatgcgtatggacttggcttgaatcactttgttccagataggctctgtaagctgaatgtcaaaactaaagaaacttgggtttggcaagagcctgattcatacccatcagaacccatctttgtttctcacccagatgccttggaagaagatgatggtgtagttctgagtgtggtggtgagcccaggagcaggacaaaagcctgcttatctcctgattctgaatgccaaggacttaagtgaagttgcccgggctgaagtggagattaacatccctgtcacctttcatggactgttcaaaaaatcttga

SEQ ID NO: 3 - protein, 533 aa, Homo sapiens
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 4 - DNA, 1672 bp, synthetic construct
  misc_feature 1..1672: note = source = /note="Description of Artificial Sequence: Syntheticpolynucleotide"
  source 1..1672: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagatcc

SEQ ID NO: 5 - DNA, 225 bp, synthetic construct
  misc_feature 1..225: note = source = /note="Description of Artificial Sequence: Syntheticpolynucleotide"
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 6 - skipped

SEQ ID NO: 7 - DNA, 141 bp, synthetic construct
  misc_feature 1..141: note = source = /note="Description of Artificial Sequence: Syntheticpolynucleotide"
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 8 - DNA, 141 bp, synthetic construct
  misc_feature 1..141: note = source = /note="Description of Artificial Sequence: Syntheticpolynucleotide"
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - skipped

SEQ ID NO: 11 - DNA, 11 bp, synthetic construct
  misc_feature 1..11: note = source = /note="Description of Artificial Sequence: Syntheticoligonucleotide"
  source 1..11: mol_type = other DNA
cctgcaggcag

SEQ ID NO: 12 - DNA, 10 bp, synthetic construct
  misc_feature 1..10: note = source = /note="Description of Artificial Sequence: Syntheticoligonucleotide"
  source 1..10: mol_type = other DNA
ttaattaagg

SEQ ID NO: 13 - DNA, 11 bp, synthetic construct
  misc_feature 1..11: note = source = /note="Description of Artificial Sequence: Syntheticoligonucleotide"
  source 1..11: mol_type = other DNA
aaagcccgggc